(19) United States
(12) Patent Application Publication
(10) Pub. No.: US 2022/0016199 A1
(43) Pub. Date: Jan. 20, 2022

(54) HYDROGEL COMPOSITION AND ASSOCIATED METHOD OF USE

(71) Applicant: Quthero, Inc., Miami, FL (US)

(72) Inventors: Milica RADISIC, Toronto (CA); Yun XIAO, Toronto (CA); Lewis REIS, Toronto (CA); Serena MANDLA, Toronto (CA)

(21) Appl. No.: 17/184,745

(22) Filed: Feb. 25, 2021

Related U.S. Application Data
(63) Continuation of application No. 15/952,004, filed on Apr. 12, 2018, now abandoned.
(60) Provisional application No. 62/484,558, filed on Apr. 12, 2017.

(51) Int. Cl. A61K 38/08; A61P 17/02; A61K 47/69; A61L 26/00; A61L 15/44

(52) U.S. Cl. CPC A61K 38/08; A61P 17/02; A61K 47/6903; A61K 9/7084; A61L 26/008; A61L 26/0066; A61L 15/44; A61K 47/6953

(57) ABSTRACT

The invention provides a composition and pharmaceutical formulation including a peptide immobilized in a hydro gel. Compositions and formulations of the invention are useful in reducing the size, severity or duration of a wound, ameliorating of one or more symptoms associated with a wound without necessarily curing the wound, or lessening in the growth or severity of a wound. Compositions and formulations of the invention are particularly useful in the treatment of a wound associated with diabetes, such as a diabetic ulcer.

### CROSS-REFERENCE TO RELATED APPLICATIONS

This application is a continuation of U.S. patent application Ser. No. 15/952,004, filed Apr. 12, 2018, which claims the benefit of U.S. Provisional Patent Application No. 62/484,558, filed on Apr. 12, 2017, the contents of each of which are incorporated herein by reference in their entirety.

### STATEMENT REGARDING FEDERALLY SPONSORED

This invention was made with government support under National Institutes of Health Grant 2R01 HL076485. The government has certain rights in the invention.

### INCORPORATION BY REFERENCE

The sequence information contained in electronic form, file name: QUTH-002/03US_Sequence_Listing.txt; size 3.6 KB; created on Feb. 23, 2021 is hereby incorporated by reference in its entirety.

### BACKGROUND

Chronic ulcers are considered a major healthcare challenge as they affect 6.5 million people in the United States. Non-healing wounds, including chronic ulcers, can be caused by a number of common diseases and medications, such as vascular insufficiency, diabetes mellitus, and local-pressure effects, which disrupt the well-orchestrated cellular and molecular interactions during the wound healing process. Specifically, diabetic foot ulcers affect 15% of people with diabetes and are a leading cause of amputation. The mechanism underlying diabetic chronic wounds remains elusive and new interventions for diabetes-impaired wound healing are needed. After almost two decades without new chemical entities approved by the Food and Drug Administration (FDA) (Regranex® was approved in 1997), it has been recognized that an optimal wound healing outcome requires a multifaceted approach that addresses different issues (e.g. persistent inflammation, insufficient angiogenesis, and impaired re-epithelialization) at once.

Keratinocytes are the major cell type in the epidermis, the outermost layer of skin. Upon injury, keratinocytes migrate from the wound edge into the wound to re-epithelialize the damaged tissue and restore the epidermal barrier. The hallmark of non-healing human wounds is non-migratory and hyper-proliferative keratinocytes, resulting in epidermis thickening at the wound edge and an absence of wound closure. Scarless embryonic wound healing and complete healing in animals with a high regenerative potential such as newts critically depend on rapid re-epithelialization.

Additionally, non-healing diabetic wounds are trapped in a state of prolonged inflammation, characterized by supra-physiological oxidative stress that can induce keratinocyte injury, dysfunction and apoptosis. It results from the excess production of reactive oxygen species (ROS) by macrophages and neutrophils, coupled with an impaired antioxidant defense capability in response to hyperglycemia. Moreover, altered extracellular matrix (ECM) composition in non-healing wounds and an enhanced ECM degradation rate due to elevated matrix metalloproteinase levels can impair keratinocyte attachment, leading to aberrant cell signaling and impaired migration.

As such, there is a clinical need for new, more effective treatments for wounds, including chronic wounds in diabetic patients. Lack of epithelial cell migration is a hallmark of non-healing wounds and diabetes often involves endothelial dysfunction. Therefore, targeting re-epithelialization, which mainly involves keratinocytes, may improve therapeutic outcomes of current treatments. for diabetic ulcers as well as for other types of chronic wounds including pressure ulcers, arterial ulcers, venous ulcers and others.

Re-epithelialization is also crucial healing process for acute wounds of partial or full thickness including trauma wounds, surgical wounds, burns, grafted skin and donor sites, scrape wounds, etc. Improving re-epithelialization in these type of wounds leads to faster healing, less scarring and less pain for patients.

### SUMMARY

To address these challenges, Applicants have developed a novel wound healing approach by 1) promoting effective keratinocyte migration, 2) protecting the wound-bed cells against oxidative stress and 3) providing a new matrix for cell attachment.

Specifically, an angiopoietin-1 derived peptide, QHREDGS (SEQ ID NO.: 1), which interacts with integrins, receptors that function in cell-adhesion and ECM-binding is used in promoting and enhancing wound healing. The QHREDGS peptide was shown to enhance endothelial cell metabolism, tube formation kinetics, and survival in response to apoptotic stimuli. QHREDGS was also shown to promote neonatal rat cardiomyocyte attachment and survival, to inhibit human induced pluripotent stem cell (hiPSC) apoptosis during cells expansion, to induce osteoblast matrix deposition and mineralization, and to have cardiac protective effects in a chitosan-collagen hydrogel both in vitro and in vivo.

QHREDGS peptide can promote keratinocyte survival and migration and thereby accelerate diabetic wound healing. QHREDGS peptide is useful as a soluble supplement or immobilized in a substrate on the survival of normal human keratinocytes upon oxidative stress by protecting keratinocytes against hydrogen peroxide stress in a dose dependent manner. Collective migration of both normal and diabetic human keratinocytes can be promoted with the QHREDGS peptide. Accelerated wound closure is also promoted with the QHREDGS peptide, primarily due to faster re-epithelialization and increased granulation tissue formation.

As such, in one aspect, the invention provides a topical composition comprising a QHREDGS peptide and a hydrogel. In certain embodiments, the hydrogel comprises at least one pharmaceutically acceptable carrier and a solvent. In certain other embodiments, the hydrogel is a polyacrylic acid hydrogel, a povidone hydrogel or a cellulose hydrogel. In certain embodiments, the hydrogel is chitosan, alginate, agarose, methylcellulose, hyaluronan, collagen, laminin, matrigel, fibronectin, vitronectin, poly-l-lysine, prozation of engineered and natural tissues, and proteoglycans, fibrin glue, gels made by decellularization of engineered and natural tissues, and a combination thereof.

In certain embodiments, the hydrogel is polyglycolic acid (PGA), polylactic acid (PLA) and combinations of PGA and PLA such as PLGA, poly F-caprolactone, polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), self-assembling peptide hydrogels, AcN-RARADADARARADADA-CNH (SEQ ID NO.: 2), polyurethanes, poly(isopropylacrylamide), poly(N-isopropylacrylamide), [poly(NIPAM)], derivatives of the same, and combinations thereof. In particular embodiments, the solvent is water.

In some embodiments of the invention, the hydrogel has an average molecular weight of about 100 Daltons (Da) to about 1,000,000 Da. In still other embodiments, the hydrogel has a viscosity from about 100 to about 10,000 cps.

In certain embodiments, QHREDGS peptide is present in the hydrogel in a concentration of about 50 μM to about 800 μM; of about 75 μM to about 750 μM; of about 100 μM to about 600 μM; or of about 150 μM to about 500 μM.

In certain embodiments, composition is in the form of a tincture, a cream, an ointment, a gel, a lotion, or an aerosol spray. In still other embodiments, the formulation is in the form of a patch or bandage. In particular embodiments, the patch or bandage comprises a backing layer and a pharmaceutical layer. In some embodiments, the backing layer is a surface layer comprising a polymer or a cellulosic; and is in the form of a film, a laminate, a woven fabric or a non-woven fabric.

In certain other embodiments, the composition further includes a stabilizer, an acidifying agent, an alkalinizing agent, an adsorbent, an aerosol propellant, an air displacement agent, an antifungal preservative, an antimicrobial preservative, an antioxidant, a binding material, a buffering agent, a carrying agent, a chelating agent, a colorant, a clarifying agent, an emulsifying agent, an encapsulating agent, a flavor ant, a humectant, a levigating agent, an oil, an ointment base, a penetration enhancer, a plasticizer, a stiffening agent, a surfactant, a suspending agent, a thickening agent, a tonicity agent, a viscosity increasing agent, a wetting agent, or a combination thereof.

In another aspect, the invention provides a method of treating a wound in a patient in need there of comprising administering to said patient a topical formulation composition according to the invention. In certain embodiments, the wound is a sore, a cold sore, a cutaneous opening, a lesion, an abrasions, a burn, a rash, an ulcer, a pressure ulcer, an arterial ulcer, a venous ulcer, a diabetes-related wound, a burn, a sun burn, an aging skin wound, a corneal ulceration wound, an inflammatory gastrointestinal tract disease wound, a bowel inflammatory disease wound, a Crohn's disease wound, an ulcerative colitis, a hemorrhoid, an epidermolysis bulosa wound, a skin blistering wound, a psoriasis wound, an animal skin wound, a proud flesh wound, an animal diabetic wound, a retinopathy wound, an oral wound (mucositis), a vaginal mucositis wound, a gum disease wound, a laceration, a surgical incision wound, a post-surgical adhesions wound, a grafted skin site or a donor skin site.

In particular embodiments of the methods of the invention, the QHREDGS peptide is present in an amount greater than about 0.1 weight % relative to the mass of the composition; greater than about 0.3 weight % relative to the mass of the composition; or greater than about 1.0 weight % relative to the mass of the composition.

In still another aspect, the invention provides a method of treating a skin condition associated with, caused by, or affected by diabetes in a patient in need there of comprising administering to said patient a topical formulation composition according to the invention. In certain embodiments, the skin condition is an atherosclerotic skin change, a bacterial infection of the skin a fungal infections of the skin, itching, NLD, Granuloma annulare, diabetic dermopathy, sclerederma, diabeticorum, APD, or a cutaneous infection.

In still another aspect, the invention provides a kit comprising a topical formulation composition according to the invention and instructions for the treatment of a wound or a skin condition. In certain embodiments the wound or skin condition is associated with, caused by, or affected by diabetes. In certain embodiments of the kit, the topical formulation composition is in a single unit dosage form. In still other embodiments of the kit of the invention, the topical formulation composition is in the form of a patch or bandage.

In yet another aspect, the invention provides a tissue scaffold comprising a QHREDGS peptide and a hydrogel. In some embodiments of the tissue scaffold of the invention, the QHREDGS peptide is present in an amount effective to induce cell growth; in an amount effective to differentiate cell growth; and/or in an amount effective to induce tissue repair. In still other embodiments of the tissue scaffold of the invention, the scaffold is configured for use in in vitro cell or tissue cultures. In particular embodiments, the scaffold is configured for use in in vivo tissue growth, grafting, repair or combinations thereof.

In particular embodiments of the tissue scaffold of the invention, the hydrogel is a polyacrylic acid hydrogel, a povidone hydrogel or a cellulose hydrogel. In other embodiments, the hydrogel is chitosan, alginate, agarose, methylcellulose, hyaluronan, collagen, laminin, matrigel, fibronectin, vitronectin, poly-l-lysine, prozation of engineered and natural tissues, and proteoglycans, fibrin glue, gels made by decellularization of engineered and natural tissues, and a combination thereof. In still other embodiments, the hydrogel is polyglycolic acid (PGA), polylactic acid (PLA) and combinations of PGA and PLA such as PLGA, poly F-caprolactone, polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), self-assembling peptide hydrogels, AcN-RARADADARARADADA-CNH, polyurethanes, poly(isopropylacrylamide), poly(N-isopropylacrylamide), [poly(NIPAM)], derivatives of the same or a combination thereof.

In particular embodiments of the tissue scaffold of the invention, the QHREDGS peptide is REDG, RLDG, REDGS, RLDGS, HREDG, HRLDG, HREDGS, HRLDGS, QHREDG, QHRLDG, QHREDVS, QHREDGS, QHRLDGS, KRLDGS, QHREDGSL, QTHRLDGSL, QHRLDGSLD, QHREDGSLD or a combination thereof. In certain embodiments, the QHREDGS peptide is immobilized in or conjugated to the hydrogel.

In some embodiments, the tissue scaffold of the invention further comprises at least one stabilizer.

In still other embodiments the tissue scaffold of the invention is in the form of a sheet, a graft, a bead, a wafer, a chip, a disc, a tube, a cylinder or a cone.

In certain embodiments, the tissue scaffold of the invention, further comprises an acidifying agent, an alkalinizing agent, an adsorbent, an aerosol propellant, an air displacement agent, an antifungal preservative, an antimicrobial agent, an antimicrobial preservative, an antioxidant, a binding material, a buffering agent, a carrying agent, a chelating agent, a colorant, a clarifying agent, an emulsifying agent, an encapsulating agent, a flavor ant, a humectant, a levigating agent, an oil, an ointment base, a penetration enhancer, a plasticizer, a stiffening agent, a surfactant, a suspending agent, a thickening agent, a tonicity agent, a viscosity increasing agent, a wetting agent, or a combination thereof. In still other embodiments, the tissue scaffold of the invention further comprises an additional active agent.

### BRIEF DESCRIPTION OF THE DRAWINGS

FIGS. 1A, 1B, 1C, 1D, and 1E demonstrate that soluble QHREDGS peptide prevents H2O2-induced cell death in human primary keratinocytes with up-regulation of Akt and MAPK phosphorylation. (1A) Kct-positive HEKs cultured in the presence or absence of different concentrations of soluble QHREDGS peptide did not incorporate significantly different amounts of BrdU, indicating similar proliferation rates in all three condition (Low: 100 μM; high: 650 μM). Scale bar=50 μm. n=3-4. (1B) Hydrogen peroxide treatment regimen for HEKs in the presence or absence of QHREDGS peptide. HEKs were allowed to attach for 2 h and serum-starved overnight. For the peptide groups, HEKs were pre-conditioned with QHREDGS peptide for 2 h and then treated with 500 μM hydrogen peroxide in the presence or absence of the peptide. (1C) HEK survival after hydrogen peroxide treatment was determined by the EarlyTox Cell Integrity assay. The QHREDGS peptide protected HEKs against H₂O₂-induced cell death in a dose-dependent manner (scale bar=200 μm). One representative experiment is shown of n=3 independent experiments with 9 replicates for each condition in one experiment. (1D and 1E) Immunoblotting with phosphorylated Akt or MAPK_{p42/44} and Akt or MAPK_{p42/44} antibodies showed transient activation of Akt and MAPK_{p42/44} pathways signaling under H₂O₂ stress. GAPDH was used as a loading control. The presence of QHREDGS peptide in the culture medium up-regulated Akt and MAPK_{p42/44} phosphorylation. n=4 independent experiments and each experiment performed in duplicates or triplicates. Data presented as mean±SD. * indicates P<0.05.

FIGS. 2A, 2B, 2C, 2D, 2E, 2F, and 2G demonstrate that immobilized QHREDGS peptide in chitosan-collagen films promotes human neonatal primary keratinocytes survival and migration. (2A) Quantification of the amount of QHREDGS peptide immobilized within chitosan-collagen films. n=3. (2B) HEK attachment on the chitosan-collagen films in the presence or absence of conjugated QHREDGS peptide. Image analysis showed no difference in the number of attached HEKs (stained with DAPI) among the three groups (scale bar=200 μm). n=3 independent experiments and each experiment performed in triplicates. (2C) Immunoblotting with anti-phosphorylated Akt or MAPK_{p42/44} and anti-Akt or MAPK_{p42/44} showed up-regulation of MAPK_{p42/44} and Akt activation during HEK attachment. GAPDH was used as a loading control. n=3 independent experiments and each experiment performed in duplicates. (2D) Experimental timeline. Cells were harvested for attachment Western blotting 2 h after seeding. Four hours after seeding, H₂O₂ was applied and the cells were harvested 15 min later for Western blotting. Live/dead staining was performed after 2 h of H₂O₂ treatment. (2E) HEK survival after hydrogen peroxide treatment was determined by the EarlyTox Cell Integrity assay. QHREDGS peptide in the chitosan-collagen film protected HEKs against H₂O₂-induced cell death in a dose-dependent manner. One representative experiment is shown of n=3 independent experiments with four replicates for each condition in one experiment. (2F) Immunoblotting showed up-regulation of the MAPK_{p42/44} and Akt phosphorylation in HEKs under H₂O₂ stress at 15 min. GAPDH was used as a loading control. n=3 and each experiment performed in duplicates. (2G) Representative examples of HEK wounding experiments on chitosan-collagen films in the presence or absence of conjugated QHREDGS peptide (scale bar=200 μm). HEK migration on the QHREDGS-immobilized films was accelerated compared to the control peptide-free films. One representative experiment is shown of n=3 independent experiments with six replicates for each condition in one experiment. Data presented as mean SD. * indicates P<0.05.

FIGS. 3A, 3B, 3C, 3D, 3E, and 3F demonstrate that immobilized QHREDGS peptide in chitosan-collagen films promotes diabetic adult human primary keratinocyte survival and migration. (3A) DHEK attachment on the chitosan-collagen films in the presence or absence of immobilized QHREDGS peptide. Image analysis showed no difference in the number of attached DHEKs (stained with TO-PRO) among the three groups (scale bar=200 μm). n=3 independent experiments and each experiment performed in triplicate. (3B) Immunoblotting showed up-regulation of MAPK_{p42/44} and Akt activation during DHEK attachment. GAPDH was used to ensure even loading. n=3 independent experiments and each experiment performed in duplicates. (3C) Experimental timeline. Cells were harvested for attachment Western blotting 2 h after seeding. Four hours after seeding, H₂O₂ was applied and the cells were harvested 15 min later for Western blotting. Live/dead staining was performed after 2 h of H₂O₂ treatment. (3D) DHEK survival following hydrogen peroxide treatment was determined by the EarlyTox Cell Integrity assay. QHREDGS peptide in the chitosan-collagen film protected DHEKs against H₂O₂-induced cell death in a dose-dependent manner. n=4-6 and each experiment performed with at least six replicates for each condition. (3E) Representative immunoblots of phosphorylated and total MAPK_{p42/44} or Akt. Quantification of immunoblotting revealed up-regulation of the Akt and MAPK_{p42/44} activation of DHEKs under H₂O₂ stress. GAPDH was used as a loading control. n=3 independent experiments and each experiment performed in duplicates. (3F) Representative examples of DHEK wounding experiments on chitosan-collagen films with or without immobilized QHREDGS peptide (scale bar=200 μm). DHEK migration on the films with QHREDGS peptide was accelerated compared to the peptide-free control. n=3 independent experiments and each experiment performed with at least four replicates for each condition. Data presented as mean±SD. * indicates P<0.05.

FIGS. 4A, 4B, 4C, and 4D demonstrate that a low dose of QHREDGS peptide immobilized to chitosan-collagen hydrogel is sufficient to promote wound healing in db/db diabetic mice. (4A) Representative images of the 8-mm full-thickness dorsal wounds on db/db diabetic mice. (4B) Representative gross images of the initial wounds on day 0 (D0) and the wounds at 14 days (D14) after treatment with no hydrogel (Blank), peptide-free chitosan-collagen hydrogel (Ctrl), and a low dose of QHREDGS peptide conjugated to chitosan-collagen hydrogel (Low peptide). Quantification of the wound size as a percentage of the original wound area revealed faster wound closure in the peptide-treated mice at day 8-14. n=4. (4C) Representative images of Trichrome stained tissue sections of wounds treated with Blank, Ctrl, and Low peptide on day 14. Black arrows indicate wound edges; red arrows indicate the tips of the healing epithelial tongue. The tips of the healing epithelial tongue were confirmed by pan-keratin staining as shown in the insets. Inset scale bar=50 μm. (4D) Quantification of wound size from histological samples collected 14 days after treatment. (i) Image analysis showed no significant difference among the three groups in the wound edge distance. (ii) The Low peptide treatment significantly reduced the size of epithelial gap, indicating accelerated wound closure compared with the Blank and Ctrl groups. (iii) The Low peptide treatment significantly increased the re-epithelialization percentage at the end of experiment compared with the Blank and Ctrl groups. (iv) The Low peptide treatment significantly increased the size of the granulation tissue compared to the Blank and Ctrl groups. (v) Average thickness of the epidermis within 300 m of the leading edge of the wound. The epidermal thickness in the Low peptide group was lower than the Blank and Ctrl groups. n=4. Data presented as mean±SD. * indicates P<0.05.

FIGS. 5A, 5B, and 5C demonstrate that a low dose QHREDGS peptide treatment does not affect microvessel density within granulation tissue but it affects total vessel number. (5A) Representative images of CD31-stained tissue sections of wounds treated with no hydrogel (Blank), peptide-free chitosan-collagen hydrogel (Ctrl), and low dose QHREDGS peptide conjugated chitosan-collagen hydrogel (Low peptide) on day 14. Scale bar=300 μm. (5B) Representative images of smooth muscle actin (SMA)-stained tissue sections from Blank, Ctrl, and Low peptide groups on day 14. Scale bar=300 μm. (5C) Microvessel analysis of (i) CD31-positive area percentage, (ii) microvessel density, (iii) total number of vessels in the entire granulation tissue, (iv) SMA-positive area percentage from representative images. n=4. Data presented as mean±SD. * indicates P<0.05.

FIGS. 6A, 6B, 6C, and 6D demonstrate that a high dose of QHREDGS peptide immobilized to chitosan-collagen hydrogel outperforms an approved wound healing dressing in db/db diabetic mice. (6A) Representative gross images of the initial wounds on day 0 (D0) and day 21 (D21) after treatment with no hydrogel (Blank), peptide-free chitosan-collagen hydrogel (Ctrl), ColActive® collagen dressing (Collagen) and a high dose of QHREDGS peptide conjugated to chitosan-collagen hydrogel (Peptide). (6B) Quantification of the wound size as a percentage of the original wound area revealed faster wound closure rate in the peptide-treated mice. n=5/group. (6C) Representative images of Trichrome stained tissue sections on day 21. Black arrows indicate wound edges; red arrows indicate the tips of the healing epithelial tongue. Scale bar=3 mm. (6D) Quantification of wound size from histological samples collected 21 days after treatment. (i) The Peptide treatment significantly reduced the wound edge distance compared to the Blank group. (ii) The Peptide treatment significantly reduced the size of epithelial gap compared to the other three groups, indicating accelerated wound closure. (iii) The Peptide treatment significantly increased the re-epithelialization percentage compared with the other three groups. (iv) The Peptide treatment significantly decreased the epithelial thickness compared with the other groups, to a level comparable to undamaged epithelium. n=4-5/group. Data presented as mean±SD. * indicates P<0.05.

FIGS. 7A and 7B. demonstrate that the presence of soluble QHREDGS peptide does not accelerate HEKs migration on collagen coated surfaces. (7A) Representative images of HEKs on collagen-coated substrates in EpiLife basal medium in the presence or absence of soluble QHREDGS peptide at the different times indicated. Scale bar=200 μm. (7B) Image analysis showed no difference in HEK migration over 24 h among the three groups. One representative experiment is shown of n=3 independent experiments with at least four replicates for each condition in one experiment. Data presented as mean±SD.

FIGS. 8A and 8B demonstrate that the presence of the immobilized QHREDGS peptide promotes HEK attachment on chitosan-only films. (8A) Representative images of HEKs on chitosan-only films in the presence or absence of immobilized QHREDGS peptide. Scale bar=200 μm. Cell nuclei are shown in blue (DAPI). (8B) Image analysis showed an increased number of attached HEKs in the presence of the immobilized QHREDGS peptide in a dose-dependent manner. The number of attached HEKs was normalized to the number that attached to tissue culture polystyrene (TCP) in the same experiment. One representative experiment is shown of n=3 independent experiments with three replicates for each condition in one experiment. Data presented as mean±SD. * indicates P<0.05.

FIGS. 9A and 9B demonstrate that HEKs form calcium-induced adherens junctions during migration and the accelerated migration is not associated with a difference in cell density. (9A) Representative images of HEKs on Ctrl substrates (chitosan-collagen films without conjugated QHREDGS peptide) in EpiLife basal medium at different times as indicated, following an increase in calcium from 0.06 mM to 0.12 mM. Adherens junctions (green=E-cadherin) were established as early as 2 h. Scale bar=50 μm. Cell nuclei are shown in blue (DAPI). (9B) HEK cell density characterized by DAPI counterstaining at the end of migration (24 h). There was no difference in the number of HEKs on the chitosan-collagen films in the presence or absence of the immobilized QHREDGS peptide. n=3 and each experiment performed in triplicates. Data presented as mean±SD.

FIGS. 10A and 10B demonstrate that the presence of immobilized QHREDGS peptide promotes DHEK attachment on chitosan-only films. (10A) Representative images of DHEKs on chitosan-collagen films in the presence or absence of immobilized QHREDGS peptide. Scale bar=200 μm. Cell nuclei are shown in blue (DAPI). (10B) Image analysis showed an increased number of attached DHEKs in the presence of immobilized QHREDGS peptide. The number of attached DHEKs was normalized to the number that attached to tissue culture polystyrene (TCP) in the same experiment. One representative experiment is shown of n=3 independent experiments with three replicates for each condition in one experiment. Data presented as mean±SD. * indicates P<0.05.

FIGS. 11A and 11B demonstrate that DHEKs form adherens junctions during migration and the accelerated migration is not associated with a difference in cell density. (11A) Representative images of DHEKs in KGM basal medium (0.1 mM Ca2+) on chitosan-collagen films in the presence or absence of QHREDGS peptide at the end of migration (6 h). Adherens junctions (green=E-cadherin) were present in all three groups. Scale bar=50 m. Cell nuclei are shown in blue (DAPI). (11B) DHEK cell density characterized by DAPI counterstaining at the end of migration (6 h). There was no difference in the number of DHEKs on chitosan-collagen films in the presence or absence of QHREDGS peptide. n=3 and each experiment performed with four replicates. Data presented as mean±SD.

FIG. 12 is an example of wound re-epithelialized after two weeks with a single treatment of QHREDGS peptide in the chitosan-collagen hydrogel. Black arrows indicate wound edges; red arrows indicate tips of the healing epithelium tongue. Re-epithelialization was quantified at 92%.

FIGS. 13A and 13B Shows the thickness of the unwounded epidermis. (13A) Representative images of Trichrome stained tissue sections of unwounded epidermis in Blank, Ctrl and low Peptide groups after 14 days in vivo. Scale bar=50 μm. (13B) Image analysis revealed no significant difference in unwounded epidermal thickness among the three groups.

FIGS. 14A, 14B, 14C, 14D, and 14E demonstrate that QHREDGS peptide does not affect microvessel number and size within granulation tissue. Microvessel analysis (14A) lumen area, (14B) vascular area, (14C) vessel area, (14D) vessel perimeter, and (14E) vessel wall thickness within the entire granulation tissue. Data presented as mean±SD. n=4.

FIG. 15 provides a regression analysis of the wound closure. Linear regression of the wound closure indicates significantly higher wound closure rate with the high dose of the Peptide (High Peptide) immobilized hydrogel compared to the Blank, peptide-free hydrogel (Ctrl) and commercially available ColActive® collagen dressing controls (Collagen). Analysis was performed using linear region of the closure data shown in FIG. 6B and testing for the significant difference between the slopes.

FIGS. 16A and 16B show a wound that exhibited hair regrowth with high dose peptide hydrogel treatment. (16A) Gross morphology photographs at Day 0 (initial wound) and Day 21 after treatment. Scale bar in mm. (16B) Mason's trichrome staining. Arrows point to hair follicles. Scale bar 300 μm.

FIG. 17 is a picrosirus red staining of the wounds treated for 21 days with Blank, peptide-free hydrogel (Ctrl), ColActive® collagen dressing (Collagen) and high dose peptide-immobilized hydrogel (High Peptide).

FIG. 18 is a PgP9.5 staining of wounds treated for 21 days with Blank, peptide-free hydrogel (Ctrl), ColActive® collagen dressing (Collagen) and high dose peptide-immobilized hydrogel (High Peptide). Scale bars=200 μm.

FIGS. 19A, 19B, and 19C shows the vascular density in wounds treated for 21 days with Blank, peptide-free hydrogel (Ctrl), ColActive® collagen dressing (Collagen) and high dose peptide-immobilized hydrogel (Peptide). (19A) Immunostaining for CD31. (19B) Immunostaining for smooth muscle actin (SMA). Scale bars=300 μm. (19C) Angiogenesis analysis i) Microvessel density evaluated by an automated algorithm from representative images of the granulation tissue. ii) Percent area covered by the CD31+ immunostaining in representative images. iii) Percent of cells highly positive for SMA in representative images.

FIGS. 20A, 20B, and 20C show the vascular density in wounds treated for 4 or 8 days with Blank or high dose peptide-immobilized hydrogel (High Peptide). (20A) Immunostaining for CD31. (20B) Immunostaining for smooth muscle actin (SMA). Scale bars=200 μm. (20C) Angiogenesis analysis i) Microvessel density evaluated by an automated algorithm from representative images of the granulation tissue. ii) Percent area covered by the CD31+ immunostaining in representative images. iii) Percent of cells highly positive for SMA in representative images. N=3 Blank Day 4, N=4 High Peptide Day 4, N=3 Blank Day 8 (i,ii), N=4 Blank Day 8 (iii), N=4 High Peptide Day 8.Data presented as mean±SD. * indicates P<0.05.

FIG. 21 illustrates an exemplary mold used to form the pre-gelled hydrogel patch.

FIG. 22 illustrates an exemplary pre-gelled hydrogel patch as described herein.

FIGS. 23A and 23B demonstrate the application of the pre-gelled hydrogel patch. (23A) Application of the patch onto the hand. (23B) Gross morphological image of the application of the pre-gelled hydrogel patch onto the back of a full excisional wound on a mouse.

### DETAILED DESCRIPTION

### Definitions

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented or understood within the context of the compound shown and well-known rules for valence interactions.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat, a mammal such as mice, rats, and non-human primates, or a farm animal such as a horse, cow, sheep, etc. In general, in the present disclosure, the term patient refers to a human patient unless otherwise stated or implied from the context of the use of the term.

The terms “treat,” treatment,” and “treating” refer to (1) a reduction in severity or duration of a condition, (2) the amelioration of one or more symptoms associated with a condition without necessarily curing the condition, or (3) the prevention of a condition.

The terms “healing,” as in “wound healing” refers to (1) a reduction in size, severity or duration of a wound, (2) the amelioration of one or more symptoms associated with a wound without necessarily curing the wound, or (3) a lessening in the growth or severity of a wound.

The term “effective” is used to describe an amount of a compound, composition or component which, when used within the context of its intended use, effects an intended result.

The term effective subsumes all other effective amount or effective concentration terms, which are otherwise described or used in the present application.

The contents of U.S. Pat. No. 9,096,643 are incorporated herein by reference in its entirety for all purposes.

Methods of Use

In one aspect, the description provides methods of treatment that include the topical administration of at least one composition in accordance with the invention to the skin and/or a wound of a patient in need thereof. In some embodiments, for example, the method includes the topical application of a composition of the invention. In some instances, such a method results in beneficial (i.e., improved) wound healing, rate of wound closure, reduced inflammation, and/or reduced rate/amelioration of infection.

In certain embodiments, the description provides a method of treating a wound in a patient in need there of comprising administering to said patient a topical formulation comprising a biomaterial and a peptide including the amino acid sequence:
X₁ X₂ X₃ X₄X₅X₆ X₇, wherein:
X₁ is an optional residue selected from glutamine, threonine, serine or asparagine;
X₂ is an optional positively charged residue selected from histidine, arginine or lysine;
X₃ is glutamate, threonine, isoleucine, histidine, lysine, glutamine, tyrosine, valine or leucine;
X₄ is glycine or valine;
X₅ is an optional residue selected from serine, threonine, aspartic acid, isoleucine or glycine;
X₆ is an optional residue selected from leucine, valine, glutamine, glycine, isoleucine or serine; and
X₇ is an optional residue selected from aspartic acid, asparagine, valine or lysine.

In certain embodiments, the formulation comprises at least one peptide including or having an amino acid sequence of QHREDGS (SEQ ID NO.: 1), REDG (SEQ ID NO.: 3), RLDG (SEQ ID NO.: 4), REDGS (SEQ ID NO.: 5), RLDGS (SEQ ID NO.: 6), HREDG (SEQ ID NO.: 7), HRLDG (SEQ ID NO.: 8), HREDGS (SEQ ID NO.: 9), HRLDGS (SEQ ID NO.: 10), QHREDG (SEQ ID NO.: 11), QHRLDG (SEQ ID NO.: 12), QHREDVS (SEQ ID NO.: 13), QHREDGS (SEQ ID NO.: 14), QHRLDGS (SEQ ID NO.: 15), KRLDGS (SEQ ID NO.: 16), QHREDGSL (SEQ ID NO.: 17), QHRLDGSL (SEQ ID NO.: 18), QHRLDGSLD (SEQ ID NO.: 19), QHREDGSLD (SEQ ID NO.: 20) or a combination thereof. In certain embodiments, the peptide is QHREDGS (SEQ ID NO.: 1).

In any of the aspects or embodiments described herein, the formulation comprises an effective amount, e.g., a therapeutically effective amount, of the peptide.

In any of the aspects or embodiments described herein, the at least one peptide is present in an amount greater than about 0.1 weight % relative to the mass of the composition, such as from about 0.3% to about 1.0% by mass, or more.

In any of the aspects or embodiments described herein, the at least one peptide is conjugated to the biomaterial. In certain embodiments, the biomaterial is a hydrogel.

In any of the aspects or embodiments described herein, the hydrogel comprises at least one of chitosan, alginate, agarose, methylcellulose, hyaluronan, collagen, laminin, matrigel, fibronectin, vitronectin, poly-l-lysine, proteoglycans, fibrin glue, gels made by decellularization of engineered and natural tissues, and a combination thereof.

In any of the aspects or embodiments described herein, the hydrogel comprises at least one of polyglycolic acid (PGA), polylactic acid (PLA) and combinations of PGA and PLA such as PLGA, poly F-caprolactone, polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), self-assembling peptide hydrogels, AcN-RARADADARARADADA-CNH (SEQ ID NO.2), polyurethanes, poly(isopropylacrylamide), poly(N-isopropylacrylamide), [poly(NIPAM)], derivatives of the same or a combination thereof.

In any of the aspects or embodiments described herein, the hydrogel comprises at least one biomaterial and a solvent. In any of the aspects or embodiments described herein, the solvent is water. In any of the aspects or embodiments described herein, the hydrogel is a polyacrylic acid hydrogel, a povidone hydrogel or a cellulose hydrogel.

In any of the aspects or embodiments described herein, the hydrogel includes a solvent, e.g., water. Other pharmaceutically acceptable carriers and solvents may be used.

In any of the aspects or embodiments described herein, the composition as described herein is comprised or dispersed within a second biomaterial, for example, a polymer or co-polymer material, such as polyvinyl, polyacrylic, polyurethane, polyethylene or the like. In certain embodiments, the second biomaterial is woven or a non-woven layer or multi-layer article that comprises or includes the peptide-hydrogel as described herein.

In any of the aspects or embodiments described herein, the hydrogel (i.e., the hydrogel material) has an average molecular weight of about 100 Daltons (Da) to about 1,000,000 Da.

In certain embodiments, the hydrogel has a viscosity from about 100 to about 10,000 cps.

In any of the aspects or embodiments described herein, the wound is external to the body cavity of the patient, for example, external surface wound or wound to the epithelium lining the gastrointestinal system or airways.

In certain embodiments, the wound is at least one of a sore, a cold sore, a cutaneous opening, a lesion, an abrasions, a burn, a rash, an ulcer, a pressure ulcer, an arterial ulcer, a venous ulcer, a diabetes-related wound, a burn, a sun burn, an aging skin wound, a corneal ulceration wound, an inflammatory gastrointestinal tract disease wound, a bowel inflammatory disease wound, a Crohn's disease wound, an ulcerative colitis, a hemorrhoid, an epidermolysis bulosa wound, a skin blistering wound, a psoriasis wound, an animal skin wound, a proud flesh wound, an animal diabetic wound, a retinopathy wound, an oral wound (mucositis), a vaginal mucositis wound, a gum disease wound, a laceration, a surgical incision wound, a post-surgical adhesions wound, a grafted skin site, or a donor skin site.

In still other embodiments, the invention relates to the treatment of a skin condition associated with, caused by, or affected by diabetes; including, but not limited to atherosclerotic skin changes, bacterial and fungal infections of the skin, itching, NLD, Granuloma annulare, diabetic dermopathy, sclerederma, diabeticorum, APD, and cutaneous infections.

Peptide and Mechanism of Action

In any of the aspects or embodiments described herein, the compositions or formulations as described herein include a non-naturally occurring peptide as described in U.S. Pat. No. 9,096,643 which is incorporated herein by reference in its entirety.

In any of the aspects or embodiments described herein, the compositions as described herein comprise at least one peptide including or having the amino acid sequence of QHREDGS (SEQ ID NO.: 1), REDG (SEQ ID NO.: 3), RLDG (SEQ ID NO.: 4), REDGS (SEQ ID NO.: 5), RLDGS (SEQ ID NO.: 6), HREDG (SEQ ID NO.: 7), HRLDG (SEQ ID NO.: 8), HREDGS (SEQ ID NO.: 9), HRLDGS (SEQ ID NO.: 10), QHREDG (SEQ ID NO.: 11), QHRLDG (SEQ ID NO.: 12), QHREDVS (SEQ ID NO.: 13), QHREDGS (SEQ ID NO.: 14), QHRLDGS (SEQ ID NO.: 15), KRLDGS (SEQ ID NO.: 16), QHREDGSL (SEQ ID NO.: 17), QHRLDGSL (SEQ ID NO.: 18), QHRLDGSLD (SEQ ID NO.: 19), QHREDGSLD (SEQ ID NO.: 20) or a combination thereof.

In any of the aspects or embodiments described herein, the formulation or composition as described herein includes a peptide including or having the amino acid sequence QHREDGS peptide (SEQ ID NO.: 1).

QHREDGS (SEQ ID NO.1) is an angiopoietin-1 derived peptide, which interacts with integrins, receptors that function in cell-adhesion and ECM-binding is used in promoting and enhancing wound healing. The QHREDGS peptide enhances endothelial cell metabolism, tube formation kinetics, and survival in response to apoptotic stimuli. QHREDGS was also shown to promote neonatal rat cardiomyocyte attachment and survival, to inhibit human induced pluripotent stem cell (hiPSC) apoptosis during cells expansion, to induce osteoblast matrix deposition and mineralization, and to have cardiac protective effects in a chitosan-collagen hydrogel both in vitro and in vivo.

The QHREDGS can promote keratinocyte survival and migration and thereby accelerate diabetic wound healing. QHREDGS peptide is useful as a soluble supplement or immobilized in a substrate on the survival of normal human keratinocytes upon oxidative stress by protecting keratinocytes against hydrogen peroxide stress in a dose dependent manner. Collective migration of both normal and diabetic human keratinocytes can be promoted with the QHREDGS peptide.

Accelerated wound closure is also promoted with the QHREDGS peptide, primarily due to faster re-epithelialization and increased granulation tissue formation.

In certain aspects, the description provides a biomaterial in which a peptide as described herein, e.g., QHREDGS peptide, is immobilized. In certain embodiments, the biomaterial is a hydrogel for use in wound healing.

In some embodiments, the QHREDGS peptide is a soluble QHREDGS peptide.

In some embodiments, the QHREDGS peptide may be linear, cyclic, cross-linked or immobilized as long as the cell-protective activity of the peptide is retained. In addition, the peptide may form a broad U-shape to assume the native structural characteristics of this peptide as it exists in angiopoietin 1.

In still other embodiments, the QHREDGS peptide may include modifications which do not substantially affect the U-shape of the core residues so as to retain the cell-protective activity of the peptide, e.g integrin-binding activity. For example, the peptide may be modified to include one or more additional amino acid residues at either the C- or N-termini, or to include a terminal protecting group that may function to stabilize the peptide, protect the peptide from undesirable degradation or improve the activity thereof. Any chemical group which serves to protect peptide ends may be used, Useful N-terminal protecting groups include, for example, lower alkanoyl groups of the formula R—C(O)— wherein R is a linear or branched lower alkyl chain comprising from 1-5 carbon atoms. Examples of N-terminal protecting groups include the acetyl group and amino acid analogues lacking the amino function. Examples of suitable carboxyl terminal protecting groups include, for example, ester-forming alkyl groups, particularly lower alkyl groups such as methyl, ethyl and propyl, as well as amide-forming amino functions such as primary amine (—NH2), as well as monoalkylamino and dialkylamino groups such as methylamino, ethylamino, diethylamino, diethylamino, methylethylamino and the like C-terminal protection can also be achieved using a decarboxylated amino acid analogue, such as agmatine. Of course, N- and C-protecting groups of even greater structural complexity may alternatively be incorporated, if desired.

In yet other embodiments, the QHREDGS peptide may also be modified at one or more of its core amino acid residues, for example, to include a derivatized R-group. Suitable modifications include those which may stabilize the U-shape of the peptide, to optimize the activity thereof, or which function to protect the peptide from degradation.

The amount of peptide or combination of peptides as described herein e.g., QHREDGS peptide, in the biomaterial is not particularly limited and can be adjusted based by one of ordinary skill in the art based on the severity of the wound and other factors generally considered by the skilled artisan. In certain embodiments of the invention, the concentration of peptide or combination of peptides present in the biomaterial is from about 10 μM to about 1000 μM. In still other embodiments, the peptide or combination of peptides are present in the biomaterial is from about 50 μM to about 800 μM; about 75 μM to about 750 μM; about 100 μM to about 600 μM; or about 150 μM to about 500 μM.

Compositions, Immobilization and Hydrogels

In one embodiment, the invention provides, a composition comprising a peptide as described herein dispersed in or immobilized on a biomaterial, for use in treatment of a skin condition and/or would healing.

Suitable biomaterials for this purpose will generally be at least one of: i) biocompatible (i.e., any material synthetic or naturally occurring that is non-toxic to the subject), ii) biodegradable, and iii) mechanically stable enough to withstand the environment into which they are administered. Suitable biomaterials may be pre-formed films, or three-dimensional porous or fibrous scaffolds. The biomaterials may also be injectable, so that they can be applied with a syringe in a minimally invasive manner. Examples of suitable biomaterials include, but are not limited to, natural biomaterials such as polysaccharides, e.g. chitosan, alginate, agarose, methylcellulose, hyaluronan, collagen (e.g. collagen I, collagen II and collagen IV), laminin, matrigel, fibronectin, vitronectin, poly-l-lysine, proteoglycans, fibrin glue, gels made by decellularization of engineered and natural tissues as well as embryoid bodies. Also included as suitable biomaterials are synthetic biomaterials such as polyglycolic acid (PGA), polylactic acid (PLA) and combinations of PGA and PLA such as PLGA, poly .epsilon.-caprolactone, polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), self-assembling peptide hydrogels such as AcN-RARADADARARADADA-CNH (but not limited to this sequence), polyurethanes, poly(isopropylacrylamide), and poly(N-isopropylacrylamide)[poly(NIPAM)]. Combinations of any of these materials may also be used as well as chemically modified forms thereof such as carboxylated or aminated forms.

In a particular embodiment, the composition comprises a QHREDGS peptide and at least one hydrogel composed of at least one biomaterial and a solvent. As examples of suitable biomaterials, non-limiting mention is made of glycerol, propylene glycol, polyethylene glycol, chitosan, alginate, agarose, polyethers, polyesters, methylcellulose, hyaluronan, collagen, laminin, matrigel, fibronectin, vitronectin, poly-l-lysine, proteoglycans, fibrin glue, gels made by decellularization of engineered and natural tissues, or a combination thereof. In certain embodiments, the hydrogel comprises or is polyglycolic acid (PGA), polylactic acid (PLA) and combinations of PGA and PLA such as PLGA, poly ε-caprolactone, polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), self-assembling peptide hydrogels, AcN-RARADADARARADADA-CNH (SEQ ID NO.2), polyurethanes, poly(isopropylacrylamide), poly(N-isopropylacrylamide), [poly(NIPAM)], derivatives of the same or combinations thereof.

In certain embodiments, the biomaterial is a hydrogel. In additional embodiments, a suitable solvent is water. Other biomaterials and solvents may be used.

In still additional embodiments, the composition is comprised or dispersed within a second biomaterial, for example, a polymer or co-polymer material, such as polyvinyl, polyacrylic, polyurethane, polyethylene or the like. In certain embodiments, the second biomaterial is woven or a non-woven layer or multi-layer article that comprises or includes the peptide-hydrogel as described herein.

In certain embodiments, the composition of a QHREDGS peptide can be a composition as described in U.S. Pat. No. 9,096,643 which is incorporated herein by reference in its entirety.

In some embodiments, the compositions include at least one water-based hydrogel. As non-limiting examples of such hydrogels, mention is made of hydrogels prepared from polyacrylic acids, povidones, celluloses, and aloe. In some embodiments, a carboxy-methyl-cellulose hydrogel is used. Of course other hydrogels may also be used in accordance with the present disclosure.

In particular embodiments, the biomaterial is chosen from polymers, such as water-soluble polymers, polymers of neutral charge, or water-soluble polymers of neutral charge. The biomaterial may also be considered by the FDA to be generally regarded as safe (GRAS). As examples of biomaterials which may be used in accordance with the present disclosure, non-limiting mention is made of hydrogels, including cellulose containing hydrogels such as carboxy-methyl-cellulose (CMC). In some embodiments of the present disclosure, the at least one biomaterial also includes at least one of water, glycerol, and mixtures thereof.

The average molecular weight of the hydrogel or the biomaterial may range, for example, from about 100 Daltons (Da) to about 1,000,000 Da, such as from about 500,000 Da to about 1,000,000 Da.

The viscosity of the biomaterial may also be chosen to suit a desired application. For example, the viscosity of the biomaterial may range from greater than 0 to about 10,000 centipoise (cps) or more, such as from about 100 to about 10,000 cps, from about 500 to about 5,000 cps, or even from about 1000 to about 3000 cps. In some embodiments, the biomaterial is a high viscosity CMC that exhibits a viscosity ranging from about 1,500 to about 3,000 cps, as measured from a 1% solution of CMC in water at 25° C. In many instances, the viscosity of the biomaterial is both concentration and temperature dependent. That is, the viscosity may decrease as temperature increases, and vice versa. Similarly, the viscosity may decrease as concentration decreases, and vice versa.

In some embodiments, the compositions of the present disclosure also include at least one stabilizer. Such stabilizers may serve a variety of purposes. For example, stabilizers may be added to the compositions of the present disclosure for the purpose of buffering the pH and/or the viscosity of the biomaterial (e.g., a hydrogel) in the presence of various metal salts. The stabilizer may be natural or synthetic, and is optionally biodegradable and/or bioerodable. Non-limiting examples of pH stabilizers that are suitable for use in accordance with the present disclosure include buffering salts and organic chemical compounds such as triethanolamine, often abbreviated as TEA, which is both a tertiary amine and a tri-alcohol. Citric acid is also suitable for use in the present disclosure as a pH stabilizer.

The QHREDGS peptide can be immobilized in or conjugated to the hydrogel by any means known in the art including, but not limited to, solvent casting. In particular embodiments, the hydrogel and the QHREDGS peptide are conjugated to using 1-ethyl-3-(3-dimethylaminopropyl) carbodiimide (EDC) chemistry. In such embodiments, the hydrogel and the QHREDGS peptide are dissolved in a solvent, such as saline, optionally containing a phosphate buffer. The dissolved materials are mixed with EDC and N-hydroxysulfosuccinimide (S-NHS) and reacted. After conjugation, the materials can be worked up using standard procedures and used to prepare a film or other composition for administration.

Pharmaceutical Compositions and Scaffolds

In certain embodiments, the invention provides a pharmaceutical formulation comprising at least one composition of the invention as described herein.

In some embodiments, the pharmaceutical formulations further comprise at least one excipient, such as a water-soluble polymer, a surfactant, and/or another enhancer such as a pharmaceutically acceptable excipient. Non-limiting examples of pharmaceutically acceptable excipients are described in Remington's Pharmaceutical Sciences by E. W. Martin, and include cellulose, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In some embodiments, the pharmaceutical formulations also contain pH buffering reagents, and wetting or emulsifying agents.

The pharmaceutical formulations of the invention can be in the any form suitable for administration to a patient, such as in the form of an aqueous dispersion or suspension. The pharmaceutical formulations may also contain various additional ingredients, such as suspending, stabilizing and/or dispersing agents.

In some embodiments, the pharmaceutical formulations are in the form of a controlled-release formulation.

The compositions of the present disclosure may also include at least one excipient. The at least one excipient may be chosen, for example, from surfactants (cationic, anionic, or neutral), surface stabilizers, and other enhancers, such as preservatives. Non-limiting examples of surfactants that may be used in accordance with the present disclosure include nonionic surfactants such as a polysorbate surfactant (e.g., polysorbate 20 (Tween 20™), and polysorbate 80 (Tween 80™)). In some embodiments, the compositions of the present disclosure contain multiple pH stabilizers so as to form a pH buffering system within the composition. As an example of a preservative that may be added to the compositions of the present disclosure, non-limiting mention is made of glycerol, which may act as a preservative at certain concentrations.

The compositions of the present disclosure may also include at least one emulsifier. Non-limiting examples of suitable emulsifiers include, phospholipids, propylene glycol, polysorbate, poloxamer, and glyceryl monostearate. Of course, other known pharmaceutical emulsifiers may be used.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃);

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobials (examples include but are not limited to silver, bismuth, chlorhexidine, polyhexamethylene biguanide (PHMB), hypochlorous acid/sodium hypochlorite, crystal violet, ozone, and antibiotics (e.g., bacitracin));

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate);

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);

chelating agents (examples include but are not limited to edetate disodium and edetic acid);

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate);

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

The compositions of the present disclosure may be in any form suitable for topical application to the skin and/or a wound. For example, the compositions may be in the form of a solution such as a hydrogel, a tincture, a cream, an ointment, a gel, a lotion, and/or an aerosol spray.

The compositions of the present disclosure may be in the form of a topical dermatologic treatment. For example, the compositions disclosed herein may be in the form of a cleansing agent, an absorbent, an anti-infective agent, an anti-inflammatory agent, an emollient (skin softener), and a keratolytic (i.e., an agent that softens, loosens, and facilitates exfoliation of the squamous cells of the epidermis).

In particular embodiments, the pharmaceutical formulation of the invention are adhered to a backing or dressing for application as a patch or bandage. In such embodiments, the pharmaceutical patch according to the invention comprises a backing layer and a pharmaceutical layer. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

The term “pharmaceutical layer” as used herein refers to any layer comprising a composition of the invention or any layer comprising a QHREDGS peptide and a hydrogel as defined herein.

The term “backing layer” as used herein refers to any layer that represents the surface layer after the application of the pharmaceutical patch. This definition includes permanent backing layer commonly used for pharmaceutical patches as well as thin non-removable films that are typically used in thin flexible patches.

In a preferred embodiment, the backing layer comprises one or more polymers selected from the group consisting of polyurethanes, polyester elastomers, polyether block amides, polyacrylates, ethylene vinyl acetates, ethylene acrylate copolymers, ionomer resins, polyvinyl chloride, polyvinylidene chloride, polyesters and polyolefins, such as polyethylene; polyolefins, in particular polyethylene, polyesters, ethylene vinylacetate copolymers and polyurethanes are particularly preferred. Other materials which can be used to form the backing include, but are not limited to, modified cellulosics such as cotton, rayon, ramie and the like; modified polyolefins such as low density polyethylene and polypropylene, modified polyesters and modified poly(acrylonitriles). Examples of particular materials of these types include oxidized cellulosics; phophorylated cellulosics; carboxymethylated cellulosics; succinylated cellulosics; grafts of polyolefins such as polypropylene with polyacrylics such as polyacrylic acid, hydrolyzed poly(acrylamides), polyacrylates, and poly(acrylonitriles); grafts of cellulosics with polyacrylics such as polyacrylic acid, hydrolyzed poly(acrylamides), polyacrylates and poly(acrylonitriles); sulfonated polyolefins; partially hydrolyzed poly(acrylonitriles) and partially hydrolyzed polyesters.

The backing layer may be a non-woven fabric or a laminate. In certain embodiments, the backing layer comprises a polymer film, such as a polyester film, and a heat seal layer.

The thickness of the backing layer is not particularly limited. Preferably, the backing layer has a thickness within the range of from 0.1 to 5000 μm; from 0.5 to 1000 μm; from 1 to 750 μm; from 5 to 500 μm; or from 10 to 250 μm.

The pharmaceutical patch according to the invention optionally comprises a removable protective layer (release liner).

In certain embodiments, the removable protective layer comprises a polymer film and a silicone coating or fluoropolymer coating. In particular embodiments, the polymer film is a polyolefin, in particular polyethylene or polypropylene film or polyester, in particular polyethylene terephthalate film.

At least one stabilizer and/or at least one excipient (described previously) may be added to the biomaterial before or after combining the biomaterial with the particles. For example, a pH stabilizer such as triethanolamine may be added to the biomaterial to stabilize the pH of the final product and/or the dispersion/suspension, if a specific pH is desired. After the components are mixed, the final product is allowed to cool to room temperature. The viscosity of the final product may be controlled, for example, by controlling the amount of stabilizer and/or other components.

In an additional aspect, the description provides a tissue scaffold comprising a composition including at least one peptide as described herein and a biomaterial, wherein the peptide is immobilized to the biomaterial. In certain embodiments, the at least one peptide has the amino acid sequence QHREDGS (SEQ ID NO.:1). In certain embodiments, the biomaterial is a hydrogel. In additional embodiments, the hydrogel is a polyacrylic acid hydrogel, a povidone hydrogel or a cellulose hydrogel; chitosan, alginate, agarose, methylcellulose, hyaluronan, collagen, laminin, matrigel, fibronectin, vitronectin, poly-l-lysine, prozation of engineered and natural tissues, and proteoglycans, fibrin glue, gels made by decellularization of engineered and natural tissues, and a combination thereof; polyglycolic acid (PGA), polylactic acid (PLA) and combinations of PGA and PLA such as PLGA, poly F-caprolactone, polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), self-assembling peptide hydrogels, AcN-RARADADARARADADA-CNH, polyurethanes, poly(isopropylacrylamide), poly(N-isopropylacrylamide), [poly(NIPAM)] or a combination thereof.

In any of the aspects or embodiments described herein, the biomaterial further comprises at least one peptide having the amino acid sequence REDG (SEQ ID NO.: 3), RLDG (SEQ ID NO.: 4), REDGS (SEQ ID NO.: 5), RLDGS (SEQ ID NO.: 6), HREDG (SEQ ID NO.: 7), HRLDG (SEQ ID NO.: 8), H REDGS (SEQ ID NO.: 9), HRLDGS (SEQ ID NO.: 10), QHREDG (SEQ ID NO.: 11), QHRLDG (SEQ ID NO.: 12), QHREDVS (SEQ ID NO.: 13), QHREDGS (SEQ ID NO.: 14), QHRLDGS (SEQ ID NO.: 15), KRLDGS (SEQ ID NO.: 16), QHREDGSL (SEQ ID NO.: 17), QHRLDGSL (SEQ ID NO.: 18), QHRLDGSLD (SEQ ID NO.: 19), QHREDGSLD (SEQ ID NO.: 20) or a combination thereof.

In certain embodiments, the peptide is conjugated to the hydrogel within the tissue scaffold.

In any of the aspects or embodiments described herein, the tissue scaffold is in the form of a sheet, a graft, a bead, a wafer, a chip, a disc, a tube, a cylinder or a cone.

In any of the aspects or embodiments, the tissue scaffold further comprising an acidifying agent, an alkalinizing agent, an adsorbent, an aerosol propellant, an air displacement agent, an antifungal preservative, an antimicrobial agent, an antimicrobial preservative, an antioxidant, a binding material, a buffering agent, a carrying agent, a chelating agent, a colorant, a clarifying agent, an emulsifying agent, an encapsulating agent, a flavor ant, a humectant, a levigating agent, an oil, an ointment base, a penetration enhancer, a plasticizer, a stiffening agent, a surfactant, a suspending agent, a thickening agent, a tonicity agent, a viscosity increasing agent, a wetting agent, or a combination thereof.

In any of the aspects or embodiments, the tissue scaffold further comprises an additional active agent. In any of the aspects or embodiments, the tissue scaffold includes an QHREDGS peptide in an amount effective to induce at least one of cell growth, cell differentiation, tissue repair or a combination thereof.

In any of the aspects or embodiments described herein, the tissue scaffold is configured for use in vitro or for use in in vivo tissue growth, grafting, repair or combinations thereof.

In some embodiments, the tissue scaffold of the invention further comprises at least one stabilizer.

In still other embodiments the tissue scaffold of the invention is in the form of a sheet, a graft, a bead, a wafer, a chip, a disc, a tube, a cylininder or a cone.

In certain embodiments, the tissue scaffold of the invention, further comprises an acidifying agent, an alkalinizing agent, an adsorbent, an aerosol propellant, an air displacement agent, an antifungal preservative, an antimicrobial agent, an antimicrobial preservative, an antioxidant, a binding material, a buffering agent, a carrying agent, a chelating agent, a colorant, a clarifying agent, an emulsifying agent, an encapsulating agent, a flavor ant, a humectant, a levigating agent, an oil, an ointment base, a penetration enhancer, a plasticizer, a stiffening agent, a surfactant, a suspending agent, a thickening agent, a tonicity agent, a viscosity increasing agent, a wetting agent, or a combination thereof. In still other embodiments, the tissue scaffold of the invention further comprises an additional active agent.

Combination Therapies/Additional Active Ingredients

In any of the aspects or embodiments described herein, the described compositions and formulations can be used alone or in combination with another therapeutic agent to treat such conditions. It should be understood that the compositions and formulations of the invention can be used alone or in combination with an additional active agent, e.g., a therapeutic agent, said additional agent being selected by the skilled artisan for its intended purpose. For example, the additional agent can be a therapeutic agent art-recognized as being useful to treat the disease or condition being treated by the compositions and formulations of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations include antimicrobial agents. Such agents include, but are not limited to, silver, bismuth, chlorhexidine, polyhexamethylene biguanide (PHMB), hypochlorous acid/sodium hypochlorite, crystal violet, ozone, and antibiotics (e.g., bacitracin)).

Preferred combinations are non-steroidal anti-inflammatory drug(s) also referred to as NSAIDS which include drugs like ibuprofen. Other preferred combinations are corticosteroids including prednisolone; the well-known side-effects of steroid use can be reduced or even eliminated by tapering the steroid dose required when treating patients in combination with the S1P receptor agonists or antagonists of this invention. Non-limiting examples of therapeutic agents for rheumatoid arthritis with which a composition of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. S/T kinase inhibitors of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7 (HUMIRA™), (PCT Publication No. WO 97/29131), CA2 (REMICADE™), CDP 571, and soluble p55 or p75 TNF receptors, derivatives, thereof, (p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-iRA etc.) may be effective for the same reason. Other preferred combinations include Interleukin 11. Yet other preferred combinations are the other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

Compositions and formulations of the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, T-cell signalling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, and Mesopram. Preferred combinations include methotrexate or leflunomide and in moderate or severe rheumatoid arthritis cases, cyclosporine and anti-TNF antibodies as noted above.

Compositions and formulations of the invention may also be combined with agents, such as alemtuzumab, dronabinol, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for HCV with which a compound of formulation of the invention can be combined include the following: Interferon-alpha-2a, Interferon-alpha-2b, Interferon-alpha con1, Interferon-alpha-n1, pegylated interferon-alpha-2a, pegylated interferon-alpha-2b, ribavirin, peginterferon alfa-2b+ribavirin, ursodeoxycholic acid, glycyrrhizic acid, thymalfasin, Maxamine, VX-497 and any compounds that are used to treat HCV through intervention with the following targets: HCV polymerase, HCV protease, HCV helicase, and HCV IRES (internal ribosome entry site).

Non-limiting examples of therapeutic agents for psoriasis with which a compound of formulation of the invention can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, and sulfasalazine.

Non-limiting examples of therapeutic agents for psoriatic arthritis with which a compound of formulation of the invention can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept and efalizumab.

Non-limiting examples of therapeutic agents for restenosis with which a compound of formulation of the invention can be combined include the following: sirolimus, paclitaxel, everolimus, tacrolimus, ABT-578, and acetaminophen.

Preferred examples of therapeutic agents for SLE (Lupus) with which a compound of formulation of the invention can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of formula (I), (Ia), (Ib), or (Ic) may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of formula (I), (Ia), (Ib), or (Ic) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of formula (I), (Ia), (Ib), or (Ic) can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of formula (I), (Ia), (Ib), or (Ic) may also be used with UP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (PCT Publication No. WO 97/29131; HUMIRA™), CA2 (REMICADE™), CDP 571, TNFR-Ig constructs, (p75TNFRIgG (ENBREL™) and p55TNFRIgG (Lenercept™)).

One or more compound of formulation of the invention can be administered to a human patient by themselves or in pharmaceutical compositions where they are mixed with pharmaceutically acceptable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Kits

This invention encompasses kits which, when used by the medical practitioner, can simplify the administration of appropriate amounts of compounds of formulations of the invention to a patient.

In certain embodiments, the description provides a kit comprising one or more unit dosage forms of a composition as described herein, including, e.g., at least one peptide as described herein and a biomaterial, and instructions for use. In any of the aspects or embodiments described herein, the peptide is QHREDGS peptide (SEQ ID NO.: 1). In any of the aspects or embodiments described herein, the biomaterial is a hydrogel as described herein.

The kits as described herein can further include any of the aspects or embodiments described herein.

Kits of the invention can further comprise devices that are used to administer compounds of formulations of the invention. Examples of such devices include, but are not limited to, intravenous cannulation devices, syringes, drip bags, patches, topical gels, pumps, containers that provide protection from photodegredation, autoinjectors, and inhalers.

Kits of the invention can further comprise pharmaceutically acceptable vehicles that can be used to administer one or more active ingredients.

The invention is further described by way of the following non-limiting examples.

### EXAMPLES

Materials and Methods

For all in vitro studies, three or more independent experiments with at least three samples per condition were performed. For animal studies, treatments in each mouse were randomized, and gross morphology analysis was performed by an investigator blinded to the study groups.

Animals, Wound Model, and Treatment

The Animal Care Committee of the University of Toronto approved all described animal studies. 8 weeks old, genetically diabetic, maleBKS.Cg-Dock7^{m}+/+Lepr^{db}/J mice (db/db) (Stock 000642) were ordered from Jackson Laboratories (Bar Harbor, USA). Mice were acclimatized for one week and their blood glucose levels were tested with a glucometer (Accu-Chek® Aviva) to confirm plasma glucose levels were over 300 mg/dL the day before surgery.

The chitosan-collagen hydrogel was prepared in a similar manner as previously described. The final hydrogel consisted of 2.5 mg/ml chitosan (with or without conjugated QHREDGS peptide) and 2.5 mg/ml type I collagen neutralized by 1 N NaOH and 10×PBS. The final hydrogel solution was mixed thoroughly and kept on ice until use. For in vivo application, the pre-gel solution was warmed for about 10 min at 37° C. to initiate the gelling process and applied to the wound site with a 2312 G needle. To benchmark the peptide-hydrogel versus an FDA-approved therapy, a commercially available ColActive® collagen dressing was gifted by Covalon Technologies, Inc. Mississauga, ON.

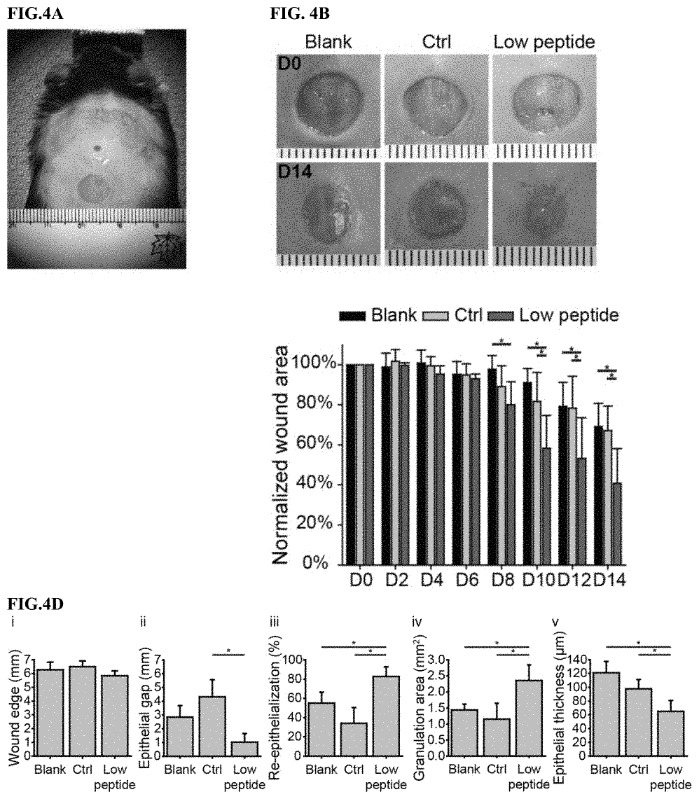

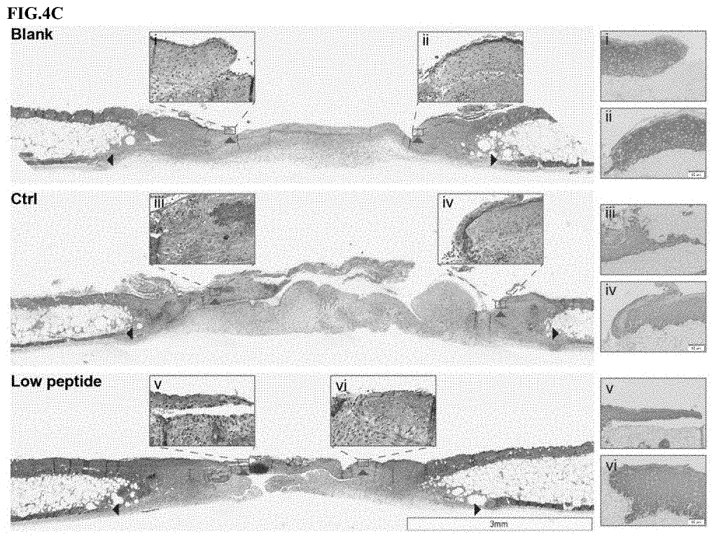

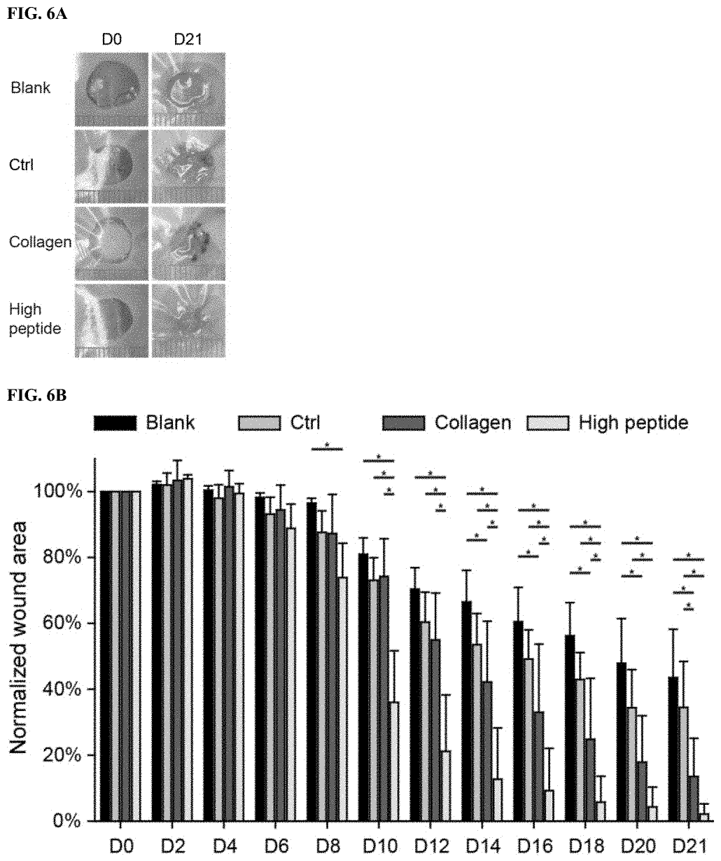

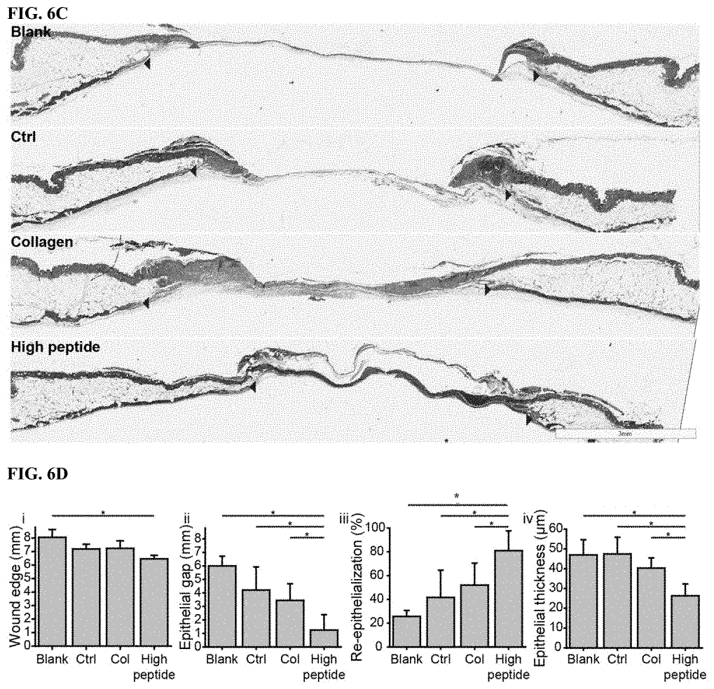

Mice were anaesthetized with inhaled isoflurane (5%) and the dorsal surface of the mouse was shaved with an electric shaver, followed by treatment with a hair removal cream (Veet®). Betadine and 70% ethanol were applied in series to the surgical site. 8 mm Biopsy punches (VWR) were used to create mid-dorsal full-thickness wounds by excising the epidermis and dermis, including the Panniculus carnosus. Either 50 μL control hydrogel without conjugated QHREDGS peptide (Ctrl), 50 μL hydrogel with Low peptide (containing a total of 2.2 nmol peptide, Low Peptide), 50 μL hydrogel with High peptide (containing a total of 5.9 nmol peptide, High Peptide), or a circular (8 mm diameter) ColActive® collagen dressing (Collagen) was applied topically to the wound beds, or the wounds were left untreated as blank (Blank). The wound beds were then covered by Tegaderm™ film (FIGS. 4B and 6A). Buprenorphine (0.03 mg/kg) was given subcutaneously before and right after the surgery as an analgesic. Thereafter, the mice were housed individually and observed every other day. Digital photographs of wounds were taken at the same distance by a camera (Canon) with a calibration scale on the side every two days. Mice were sacrificed using CO2 asphyxiation, followed by cervical dislocation, on day 4 (Blank, High Peptide), 8 (Blank, High Peptide), 14 (Blank, Ctrl, Low Peptide), or 21 (Blank, Ctrl, Collagen, High Peptide) as indicated.

Statistical Analysis

All results are presented as mean±SD. Statistical analysis was performed using SigmaPlot 11.0. Differences between experimental groups were analyzed using one-way or two-way ANOVA followed by a Tukey post hoc test for pairwise comparison. Linear regression was used to calculate wound closure rates and significant differences were determined by comparing the slopes of the fitted lines. A value of P<0.05 was considered as statistically significant.

Primary Human Keratinocytes Cell Culture

Primary neonatal human epithelial keratinocytes (HEKs) were purchased (Cascade Biologics) and cultured in EpiLife medium supplemented with EpiLife Defined Growth Supplement (EDGS) as recommended by the manufacturer (Cascade Biologics; referred to as complete medium). HEKs were cultured on surfaces coated with a coating matrix kit (Cascade Biologics) and passaged using 0.025% trypsin/EDTA (Cascade Biologics) until 70-80% confluence was reached. Third and fourth passage HEKs were used in experiments.

Diabetic human adult epithelial keratinocytes (DHEKs) from a patient (72 years old female) with type II diabetes were purchased from Lonza and cultured in KGM-Gold™ BulletKit™ medium as recommended by the manufacturer (Lonza). DHEKs were passaged using ReagentPack™ Subculture Reagents (CC-5034, Lonza) until 70-80% confluent. Second and third passage DHEKs were used in experiments.

Evaluation of Soluble QHREDGS In Vitro

The effect of the QHREDGS peptide as in the soluble form was assessed on HEKs in vitro. 24- or 96-well plates were coated with 0.05 mg/mL type I collagen (BD Biosciences) in 0.02 N acetic acid overnight and then rinsed once with phosphate buffered saline (PBS) (Lonza). HEKs were seeded in complete EpiLife medium and attached for at least 2 h. 100 μM or 650 μM QHREDGS was supplemented to the EpiLife medium as Low or High dosage, respectively.

Proliferation Assay

HEKs were seeded at a density of 1×10⁴ cells/cm² in collagen-coated 96-well plates. After 4 h, media was removed and replenished with EpiLife media supplemented with 20 μM bromodeoxyuridine (BrdU) (Sigma Aldrich) in the presence or absence of QHREDGS peptide. After incubating for 8 h, proliferating HEKs were identified by BrdU staining. Briefly, HEKs were fixed with 4% paraformaldehyde (PFA), permeablized with 0.25% triton X, treated with DNase for 30 min at 37° C., and blocked with 5% bovine serum albumin (BSA). The HEKs were then incubated with a rat anti-BrdU antibody (AbD Serotec) overnight at 4° C. followed by an anti-rat TRITC-labelled secondary antibody (Jackson Immuno Research). BrdU-positive cells were counted in 5 randomly chosen fields under 20× magnification and then normalized to the total number of cells labelled by 4′,6-diamidino-2-phenylindole (DAPI) counterstaining.

H₂O₂ Treatment of HEKs with Soluble QHREDGS Peptide

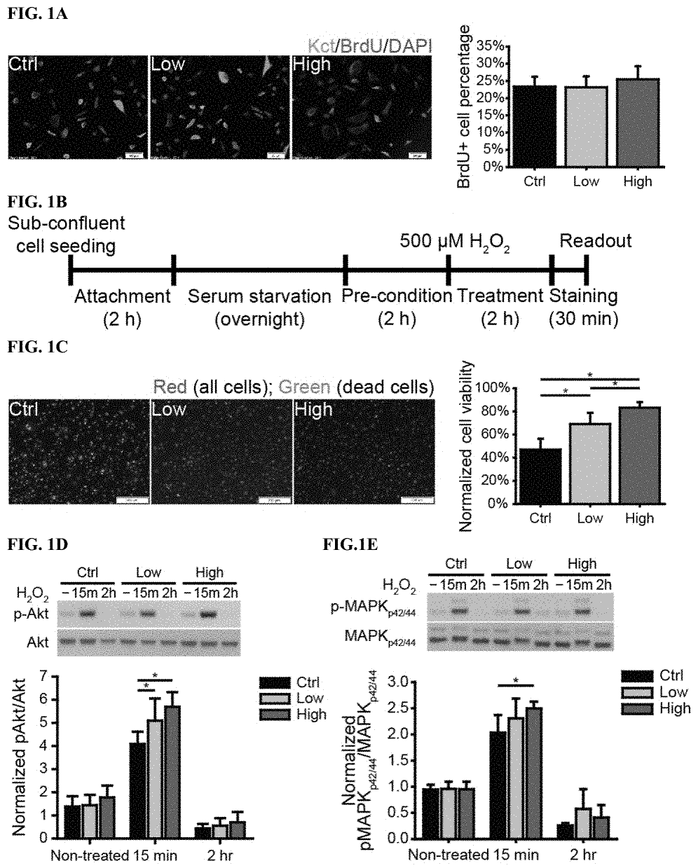

HEKs were treated by H₂O₂ as shown in FIG. 1B. HEKs were seeded at a sub-confluent density in a 96-well plate for the cell integrity assay or a 24-well plate for Western blot analysis, and serum starved overnight. The HEKs were then pretreated with soluble QHREDGS (100 μM for Low and 650 μM for High) for 2 h. The cells were then exposed to fresh EpiLife basal medium supplemented with 500 μM H₂O₂ in the presence or absence of QHREDGS peptide. For the cell integrity assay, the cells were stained using the EarlyTox™ Cell Integrity kit (Molecular Devices) after 2 h of H₂O₂ treatment. For the Western analysis, another set of wells were seeded in parallel for the Ctrl, Low and High conditions and protein samples were collected after 0, 15 min and 2 h of H₂O₂ treatment.

Conjugation of QHREDGS to Chitosan

The QHREDGS peptide was conjugated to chitosan using 1-ethyl-3-(3-dimethylaminopropyl) carbodiimide (EDC) chemistry as previously described. (84) Briefly, chitosan (UP G 113, Novamatrix) was dissolved at 20 mg/ml in 0.9% normal saline and the QHREDGS peptide at 10 mg/ml in PBS. These were then mixed with EDC and N-hydroxysulfosuccinimide (S-NHS), dissolved in PBS, to a final solution concentration of 5 mg/ml chitosan and 0.5 mg/ml of QHREDGS peptide (Low peptide group) or 3 mg/ml of QHREDGS peptide (High peptide group). In the reaction solution, the mass ratio of [EDC]/[peptide] and [S-NHS]/[EDC] were kept constant at 0.8 and 2.75, respectively. The reaction solution was left on a vortex mixer (VWR) at 650 rpm for 3 h, diluted 4× with PBS and dialyzed against distilled water for 48 h (Spectra/POR MWCO 3500, Spectrum Labs). The dialyzed solution was then filter sterilized, lyophilized for 48 h and stored at −20° C. until use.

Solvent Casting of Chitosan-Collagen Films

The chitosan samples were reconstituted at 2 mg/ml in 0.5 N acetic acid and mixed with 2 mg/ml type I collagen (BD Biosciences) to obtain a film coating solution composed of 1 mg/ml each of chitosan and collagen. 24-well plates were coated with 250 μL film coating solution per well and 96 well plates with 50 μL per well. The film coating solution was fully evaporated in a biosafety hood and the chitosan-collagen films were cast in the wells. The coated plates were rinsed three times with ample PBS before use.

Coating Validation

Fluorescently labeled peptide, FITC-QHREDGS (Biomatik), was used to validate peptide concentrations in the film. FITC-QHREDGS was substituted for regular peptide in the protocol above and all steps were protected from light. Standards of the FITC-QHREDGS in PBS were made ranging from 0.025 pg/ml to 0.5 pg/ml. After rinsing with PBS three times, the coated 96-wells were filled with 200 μl PBS and then run, with the standards in the same plate, through a fluorometer (SpectraMax i3, Molecular Devices) at excitation and emission wavelengths of 490 nm and 520 nm, respectively. The final amounts of peptide were quantified using SoftMax Pro 6.4 software.

Keratinocyte Attachment on Chitosan-Only Films

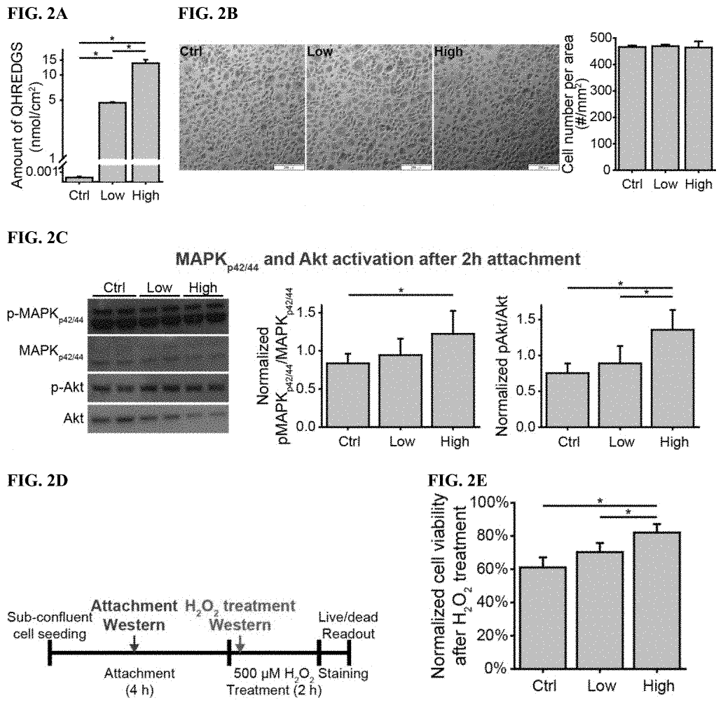

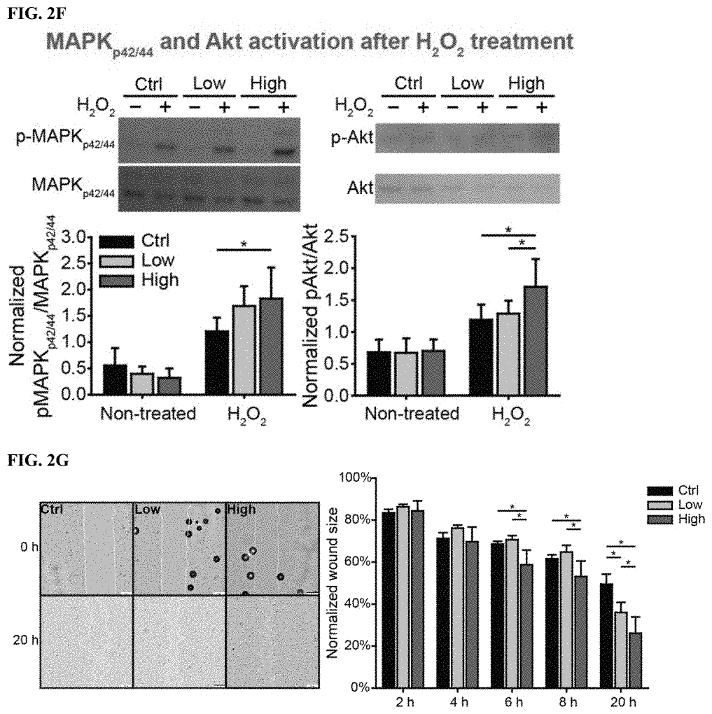

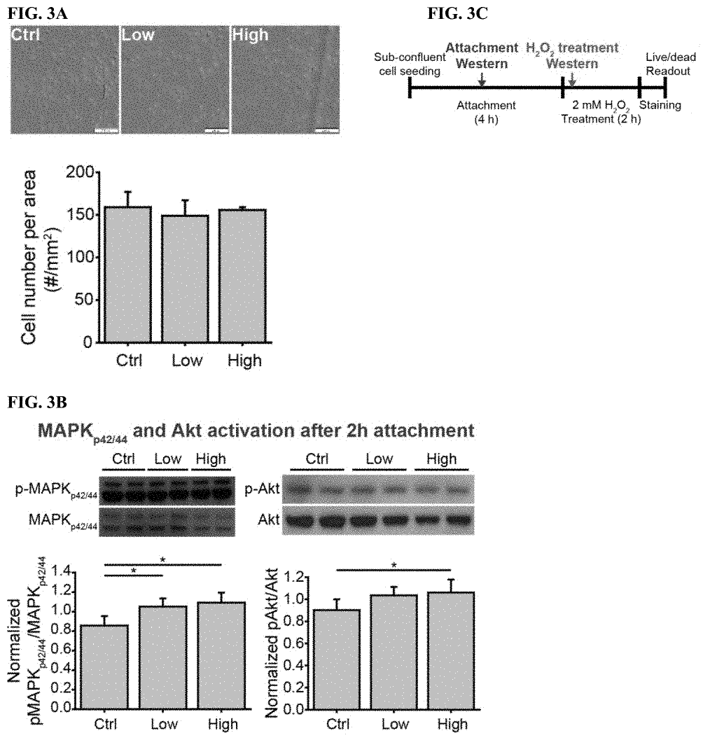

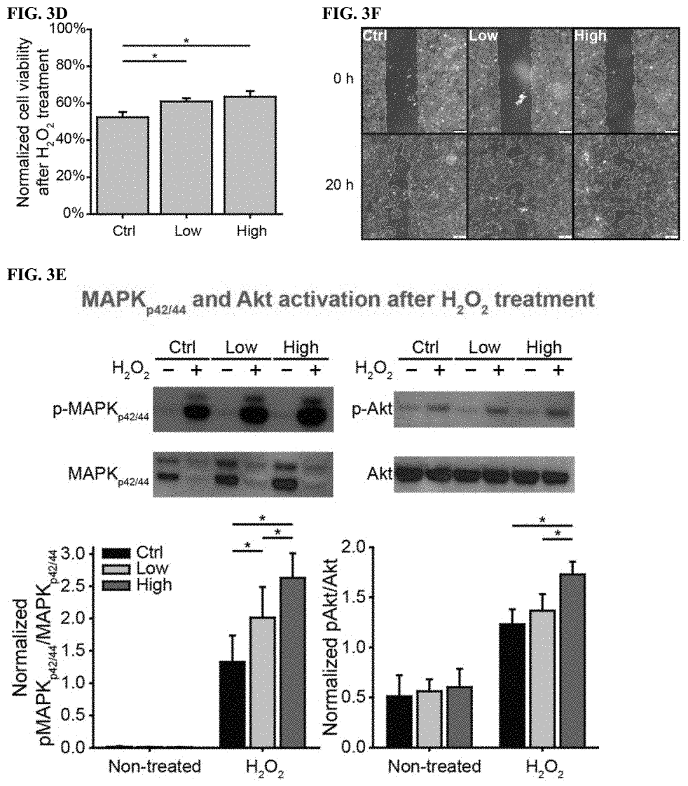

Chitosan-only films were prepared by casting 1 mg/ml chitosan with the presence or absence of conjugated QHREDGS peptide in 0.5 N acetic acid in the wells of a 96-well plate and drying in the a biosafety hood overnight. The coated plates were rinsed three times with ample PBS before use. HEKs or DHEKs were seeded in supplemented EpiLife or KGM medium and allowed to attach on the chitosan-only films. After 2 h (timeline shown in FIGS. 2D and 3C) unattached cells were carefully rinsed off using PBS and cells were then fixed with 4% paraformaldehyde. The number of attached cells were quantified using DAPI counterstaining and the cell numbers on chitosan-only films in the presence or absence of conjugated QHREDGS peptide were normalized to the number of cells attached to regular tissue culture polystyrene (TCP).

H₂O₂ Treatment of Keratinocyte on the Chitosan-Collagen Films

HEKs or DHEKs were seeded in complete EpiLife or KGM medium and allowed to attach for 4 h. HEKs were then changed to basal EpiLife medium supplemented with 500 μM H₂O₂ and DHEKs were changed to basal KBM medium supplemented with 2 mM H₂O₂ to mimic conditions of higher oxidative stress and oxidative damage in human chronic wounds. After 2 h, cells were changed to a complete medium with EarlyTox™ Cell Integrity staining reagent and subjected to cell integrity assay. Cells treated by H₂O₂ for 15 min were used for Western blotting together with the non-treated controls, as shown in the timeline, FIGS. 2D and 3C.

EarlyTox Cell Integrity Assay

The EarlyTox™ Cell Integrity Kit (Molecular Devices, R8213) is based on two nuclear dyes: live red dye is cell permeant and marks both live and dead cells (Excitation: 622 nm/Emission: 645 nm); dead green dye is cell impermeant and stains only cells with damaged outer membranes (Excitation: 503 nm/Emission: 526 nm). To avoid cell detachment, half of the medium in each 96 well was removed by micropipette and the equal volume of the staining solution with double concentration of the dyes was added into the well carefully, such that the final concentration in the well was the one suggested by the manufacturer. The plate was then incubated at 37° C. for 30 min and imaged using a SpectraMax i3 plate reader (Molecular Devices). The percentage of viable cells after H₂O₂ treatment on chitosan-collagen films in the presence or absence of QHREDGS peptide was normalized to the viability percentage of non-treated controls on the same coating film condition.

Western Blotting

Protein was isolated from keratinocytes in the 24-well plate after H₂O₂ treatment using 60-80 μL Lysis Buffer per well (10× Cell Lysis Buffer, Cell Signaling Technology; complete Mini, EDTA-free protease inhibitor cocktail tablet, Roche; in ddH2O). Proteins were separated by electrophoresis in Novex Tris-Glycine gels (Life technologies) and transferred using the iBlot (Life technologies) to a PVDF iBlot Transfer Stack (Life technologies). Membranes were probed for phospho-p44/42 MAPK (pMAPK_{p42/44}), p44/42 MAPK (MAPK_{p42/44}), phospho-Akt, Akt, or GAPDH as a loading control (Millipore). All primary antibodies were purchased from Cell Signaling unless stated otherwise. HRP conjugated goat anti-mouse or goat anti-rabbit secondary antibodies were used (DAKO). Membranes were developed with Amersham ECL or Amersham ECL Prime Western Blotting Detection Reagent (GE Healthcare) and exposed to the films. The films were scanned and densitometry was performed using ImageJ or Image Studio™ Lite (LI-COR Biosciences).

Migration Assay

Culture-inserts were purchased from Ibidi® and carefully placed on the chitosan-collagen films in 24-wells. HEKs were seeded in complete EpiLife medium at 0.3-0.4×10⁶ cells/mL with 70-100 μL per chamber and allowed to attach for 2 h. The culture-inserts were then carefully lifted and the unattached HEKs were immediately removed by rinsing twice using warm PBS. Basal EpiLife medium was then added to the wells and the wells were imaged every 2 h for 8 h and the next day using the SpectraMax i3 plate reader. Confluent HEK monolayers were wounded (time 0) and maintained for 24 h in EpiLife basal medium with 0.12 mM Ca2+. The wounds were outlined and the area at the various time points was normalized to the initial wound size (time 0). After 24 h, HEKs were fixed with 4% paraformaldehyde.

Similarly, DHEKs were seeded in complete KGM medium with CellMask Green (1000× dilution) at a density of 0.15-0.2×10⁶ cells/mL in ibidi migration chambers and allowed to attach for 2 h. Migrations were initiated by lifting the culture-inserts as mentioned above and the wells were imaged every 2 h for 6 h using the SpectraMax i3 plate reader. Confluent DHEKs were stained with CellMask Green, wounded at time 0 and maintained for 6 h in KBM basal medium. The wounds were outlined and the area at the indicated times were normalized to the initial wound size (time 0). After 6 h, DHEKs were fixed with 4% paraformaldehyde.

Calcium Increase for HEK Migration

The Ca²⁺ level in the EpiLife medium was increased from 0.06 mM to 0.12 mM to ensure HEKs migrated collectively. After initiating the HEK migration by lifting the Ibidi® culture inserts, HEKs were maintained in EpiLife basal medium supplemented with CaCl₂) at a final Ca²⁺ concentration of 0.12 mM. HEKs were fixed with 4% paraformaldehyde after exposure to increased Ca²⁺ concentration for the indicated period of time.

Immunostaining

At the end of migration, HEKs and DHEKs were fixed and stored in PBS at 4° C.

Cell monolayers were permeablized with 0.25% triton X, blocked with 5% bovine serum albumin (BSA), and then incubated with mouse anti-E-cadherin primary antibody (BD Biosciences; 1:200) overnight at 4° C. followed by goat anti-mouse Alexa 488 secondary antibody (Jackson Immuno Research; 1:400). Cell nuclei were counterstained with 4′,6-diamidino-2-phenylindole (DAPI) (Biotium; 1:100). The 24-well plates were imaged at 20× magnification using a fluorescence microscope (Olympus IX81).

Histology Analysis

Following euthanasia, the wound tissue was excised together with surrounding tissue and fixed in 10% formalin (Sigma). Tissue samples were embedded in paraffin blocks and then sliced into 5 μm-thick sections. Sections were processed and stained with hematoxylin and eosin (H&E), Masson's trichrome, or immunostained using anti-CD31, anti-smooth muscle actin (SMA), and anti-PgP9.5 (PgP9.5) antibodies at the University Health Network Pathology Research Program laboratory. Picosirious red staining was performed at the Toronto Center for Phenogenomics. Stained slides were scanned (20×) using the Aperio ScanScope XT (Aperio Technologies, USA) at the Advanced Optical Microscopy Facility (AOMF, Toronto, Canada). Picrosirius red staining for collagen was performed at the pathology laboratory of Princess Margaret Hospital. The images of scanned slides were analyzed using the Aperio ImageScope (Version 11).

In order to characterize the healing of the wounds, Masson's trichrome stained slides were scanned with a ScanScope XT whole slide scanner and measured using Aperio ImageScope (v11, Aperio Technologies). The wound edge was defined as the Panniculus carnosus muscle gap, the epithelial gap as the distance between the epithelial tongues, and the re-epithelization percentage as the ratio of epithelial gap over wound edge. The size of the granulation tissue was defined by the highly cellular tissue between epidermis and the fat/muscle tissue. The epidermal thickness was defined as the average thickness of the leading epithelial tongue (300 μm) from both ends. In order to characterize angiogenesis from CD31-stained images, an automated algorithm (Aperio ImageScope, Microvessel Analysis Algorithm) was used over the entire granulation tissue or for representative images from the granulation tissue. The SMA positive area percentage was determined from six random views (300 μm×300 μm) within the granulation tissue.

Microvessel & Positive Staining Analysis Algorithms

Scanned images of the histology slides stained with anti-CD31 antibody were analyzed using the Microvessel Analysis v1 algorithm with Aperio ImageScope (Version 11). The entire granulation tissue or representative areas were selected using a drawing tool and CD31 positive staining was thresholded by Color Deconvolution. Microvessels were thresholded using a Region Joining Parameter of 6-8 μm, a Vessel Completion Parameter of 7-10 μm, and a Vessel Area between 5 and 20000 μm². The automated algorithm calculated the Total Vessel Number, Lumen Area, Vascular Area, Vessel Area, Vessel Perimeter, and Vessel Wall Thickness (among other parameters) for each vessel within the selected granulation tissue area and output the results in histograms. For SMA+cell percent quantification, the entire granulation tissue or representative areas were selected using a drawing tool and analyzed with Positive Pixel Count v9.1 in ImageScope. Only high intensity (identified as strong positive, threshold 0-100) staining was considered as positive SMA staining.

Results

QHREDGS Peptide Prevents H₂O₂-Induced Cell Death in Human Primary Keratinocytes Via Akt and MAPK_{p42/44} Signaling

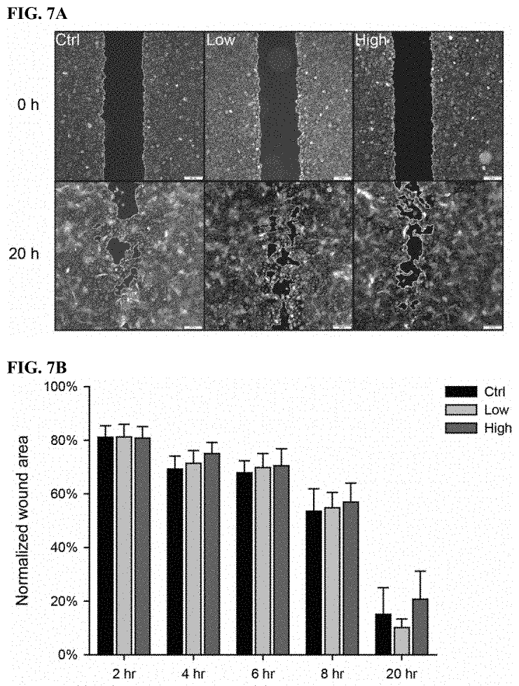

To evaluate the effect of the QHREDGS peptide on keratinocytes, normal neonatal human epidermal keratinocytes (HEKs) cultured with the soluble QHREDGS peptide at doses previously reported to be effective for endothelial cell survival weret focused on (Low: 100 μM; High: 650 μM)(14). The percentage of proliferating HEKs in the population was not affected by the presence of the QHREDGS peptide at either concentration as quantified by bromodeoxyuridine (BrdU) incorporation (FIG. 1A). No significant difference in the HEK migration rate was observed with the soluble QHREDGS peptide at either concentration (FIG. 7).

To investigate the effect of the soluble QHREDGS peptide on keratinocyte survival under oxidative stress, HEKs were pre-conditioned by incubating the cells with or without the QHREDGS peptide and then exposed the HEKs to 500 μM H₂O₂ for 2 h (FIG. 1B). An endpoint cell integrity assay showed a significant dose-dependent increase in the percentage of viable HEKs in the presence of supplemented QHREDGS (FIG. 1C).

Given that the full-length protein from which the QHREDGS peptide was derived, angiopoietin-1, is known to protect skin cells from oxidative damage and to increase the activation of the prosurvival Akt and MAPK_{p42/44} pathway, it was investigated whether improved survival upon H₂O₂ stress in the presence of the QHREDGS peptide was associated with the upregulation of Akt and MAPK_{p42/44} phosphorylation. HEKs treated with 500 μM H₂O₂ showed transient phosphorylation of both Akt (FIG. 1D) and MAPK_{p42/44} (FIG. 1E) at 15 min by Western blot analysis. Indeed, the presence of the soluble QHREDGS peptide during pre-conditioning and H₂O₂ treatment increased the phosphorylation of Akt and MAPK_{p42/44} and the increase was dose-dependent (FIGS. 1D and E).

Immobilized QHREDGS Peptide Promotes Human Primary Keratinocyte Attachment, Survival and Migration In Vitro

While it was observed increased survival without excessive proliferation in the presence of soluble QHREDGS, enhanced keratinocyte migration was not observed (FIG. 7). This motivated further optimization of the method by which the peptide was presented to the cells. Given that the QHREDGS peptide is reported to primarily function through integrin interactions and there is an ever-growing body of literature showing the increased efficacy of integrin ligands when immobilized to a matrix, the QHREDGS peptide was covalently immobilized to a chitosan-collagen hydrogel. Chitosan and collagen interact through a combination of thermal and ionic mechanisms, stabilized by polyanion (collagen) and polycation (chitosan) electrostatic interactions. Conjugation of the QHREDGS peptide to chitosan was achieved using previously described methods and chitosan-collagen films with or without immobilized QHREDGS peptide were cast in the wells of 24-well or 96-well plates. Quantification using fluorescently labelled peptide, FITC-QHREDGS, demonstrated effective immobilization in both Low (4.7±0.1 nmol/cm²) and High (13.8±1.4 nmol/cm²) peptide concentrations and absence of the peptide in the Ctrl condition (FIG. 2A). Normalized to the mass of chitosan in the films, the amount of immobilized QHREDGS peptide was 14.9±0.3 nmol/mg in the Low condition and 44.1±4.6 nmol/mg in the High condition.

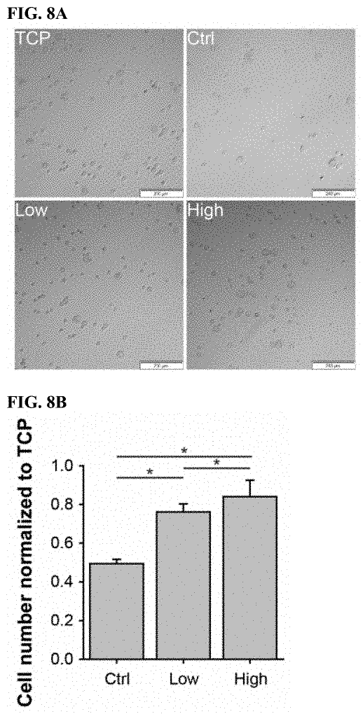

There was no significant difference in the attachment of HEKs to the various chitosan-collagen films (FIG. 2B). However, in the settings of chitosan-only films wherein adhesion was poor, the QHREDGS peptide clearly promoted HEK attachment in a dose-dependent manner (FIG. 8). This indicates that while the QHREDGS peptide can promote HEK attachment, the presence of collagen adhesion sites in the setting of the chitosan-collagen film masks this effect. Furthermore, Western blot analysis showed the increased activation of Akt and MAPK_{p42/44} during 2 h attachment on chitosan-collagen films in the presence of immobilized QHREDGS peptide (FIG. 2C).

The effect of the immobilized QHREDGS peptide on HEK survival following 500 μM H₂O₂ treatment was then investigated. HEKs were allowed to attach to the chitosan-collagen films for 4 h, then treated with H₂O₂ for 2 h (FIG. 2D). Subsequent cell integrity assessment showed an increased percentage of viable HEKs in the presence of the immobilized QHREDGS peptide (FIG. 2E). HEKs treated with 500 μM H₂O₂ and non-treated controls were also compared using Western blot analysis, wherein phosphorylation of MAPK_{p42/44} was increased in the presence of immobilized QHREDGS peptide relative to the control (FIG. 2F).

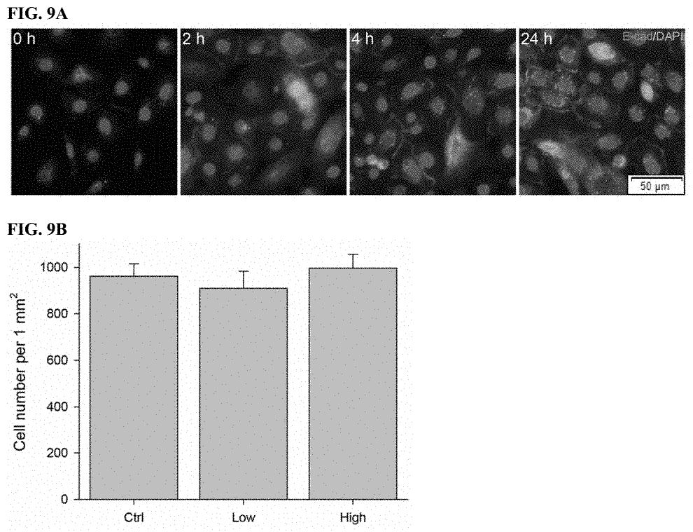

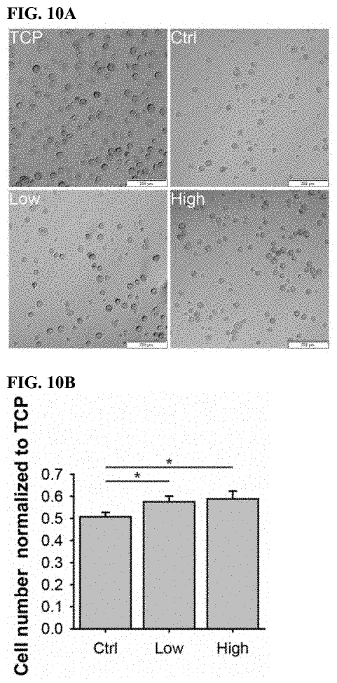

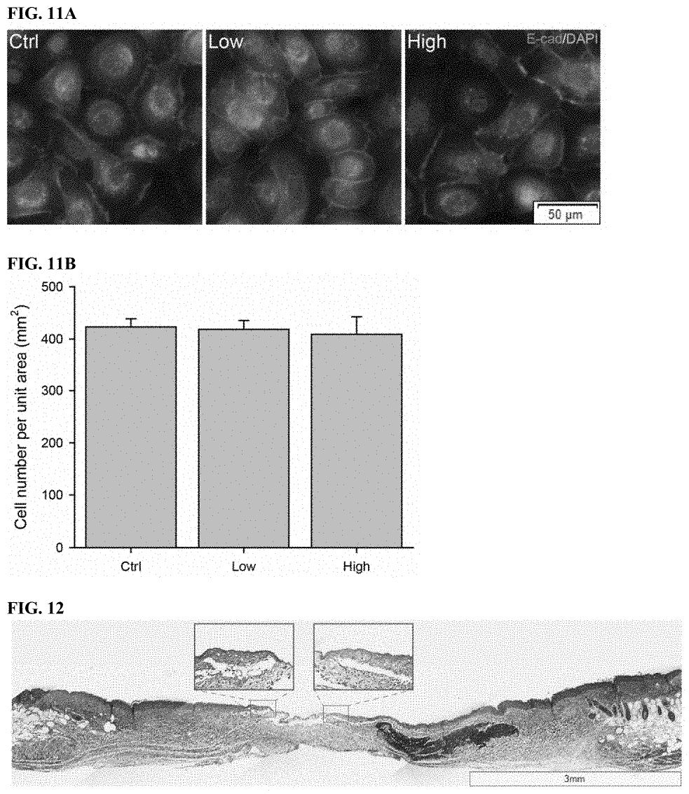

Keratinocyte migration is essential for wound healing as a wound cannot heal in the absence of re-epithelialization. (25) The effect of the immobilized QHREDGS peptide on HEK migration in 2D monolayers using an Ibidi migration assay system was assessed. Importantly, the Ca²⁺ concentration in the culture medium was increased from 0.06 mM to 0.12 mM upon initiation of the migration assay to ensure collective HEK migration (essential for wound healing), as demonstrated by the formation of E-cadherin mediated cell-cell junctions (FIG. 9A). The presence of the immobilized QHREDGS peptide accelerated collective HEK migration in a dose-dependent manner (FIG. 2G). The accelerated migration was not due to increased proliferation as there was no difference in cell density among the three groups as characterized at the migration endpoint (FIG. 9B).

Immobilized QHREDGS Peptide Promotes Diabetic Human Primary Keratinocytes Attachment, Survival and Migration In Vitro

In diabetic chronic wounds, keratinocytes experience hyperglycemia and supra-physiological oxidative stress, which challenges keratinocyte's proliferation and survival. The effect of the immobilized QHREDGS peptide on adult diabetic human epidermal keratinocytes (DHEKs) by seeding DHEKs onto chitosan-collagen films in the presence or absence of immobilized QHREDGS peptide was examined. Similar to the results with normal HEK cells, it was found that the presence of the immobilized QHREDGS peptide promoted DHEK attachment to chitosan-only films (FIG. 10) but did not affect DHEK attachment to chitosan-collagen films (FIG. 3A). Western blot analysis showed that the activation of MAPK and Akt was increased in DHEKs in the presence of the QHREDGS peptide during a 2 h attachment (FIG. 3B).

Because of prolonged inflammation, human chronic wounds experience 3- to 4-times higher oxidative stress and oxidative damage compared to acute wounds. To mimic this scenario, the effect of immobilized QHREDGS peptide on DHEK survival following a 2 h treatment with 2 mM H₂O₂ (4-times higher exposure than used for the HEK survival assay) was investigated, after allowing DHEKs to attach to the chitosan-collagen films for 4 h (FIG. 3C). Cell integrity assessment showed that DHEK survival under H₂O₂ stress was improved in the presence of immobilized QHREDGS peptide (FIG. 3D), despite the higher H₂O₂ concentration used. DHEKs treated with 2 mM H₂O₂ and non-treated controls were also compared by Western blot analysis and phosphorylation of Akt and MAPK_{p42/44} upon H₂O₂ treatment was increased in the presence of immobilized QHREDGS peptide in a dose-dependent manner (FIG. 3E).

The effect of immobilized QHREDGS peptide on DHEK migration was assessed using the Ibidi migration assay. Importantly, the Ca²⁺ concentration in KGM medium (0.1 mM) was sufficient to ensure collective DHEK migration as demonstrated by the formation of E-cadherin mediated cell-cell junctions without additionally elevating the Ca²⁺ concentration (FIG. 11A). The presence of the immobilized QHREDGS peptide also accelerated DHEK collective migration (FIG. 3F) and this was not due to cell density differences as characterized at the migration endpoint (FIG. 11B).

QHREDGS-Immobilized Hydrogel Promotes Wound Healing in Db/Db Diabetic Mice

Whether the QHREDGS peptide immobilized to the chitosan-collagen hydrogel could accelerate wound healing in diabetic mice was investigated. This hydrogel system was chosen as a delivery vehicle because of its rapid gelation under physiological conditions and its persistence for a period of 3 weeks in vivo. In vivo biocompatibility was also demonstrated in previous myocardial infarction model studies. Therefore, only one application of the hydrogel onto the wounds was needed for the 2 week study. A full-thickness excision wound (FIG. 4A) was created on eight weeks old, male BKS.Cg-Dock7^{m}+/+Lepr^{db}/J mice (db db). This model was selected because the animal is leptin receptor deficient and represents a type II diabetes model characterized by hyperglycemia, obesity, hyperinsulinemia, and impaired wound healing. Moreover, this strain heals wounds primarily by granulation tissue formation rather than by contraction.

Quantification using fluorescently labelled peptide demonstrated that in reconstituted chitosan solutions, the amount of conjugated QHREDGS peptide was 17.5±2.2 nmol/mg_{(chitosan)} in Low conditions and 41.5±1.4 nmol/mg_{(chitosan)} in High conditions. This was converted to a peptide concentration in the final chitosan-collagen hydrogel of 43.8±4.4 M in Low conditions and 103.8±3.5 μM in High conditions. With a view to future clinical translation, the Low condition in the in vivo studies was tested to minimize the amount of peptide applied to the wound. A single application of 50 μL Low chitosan-collagen hydrogel (2.2 nmol immobilized QHREDGS peptide; Low Peptide) was applied to the wound. The chitosan-collagen hydrogel alone without the peptide (Ctrl) and a no hydrogel/no peptide (Blank) were used as controls. A secondary dressing, Tegaderm™ film, was applied on top of the wound with or without the hydrogel, to maintain a moist environment. As shown in FIG. 4B, the presence of immobilized QHREDGS in the hydrogel resulted in significantly smaller wounds on day14 compared to the controls. Image analysis of the wound gross morphology performed by an investigator blinded to the study groups demonstrated faster wound healing in the Low Peptide group starting on day8. Administration of the chitosan-collagen hydrogel without the immobilized QHREDGS peptide (Ctrl) had no significant effect on the wound closure rate compared to the Blank controls.

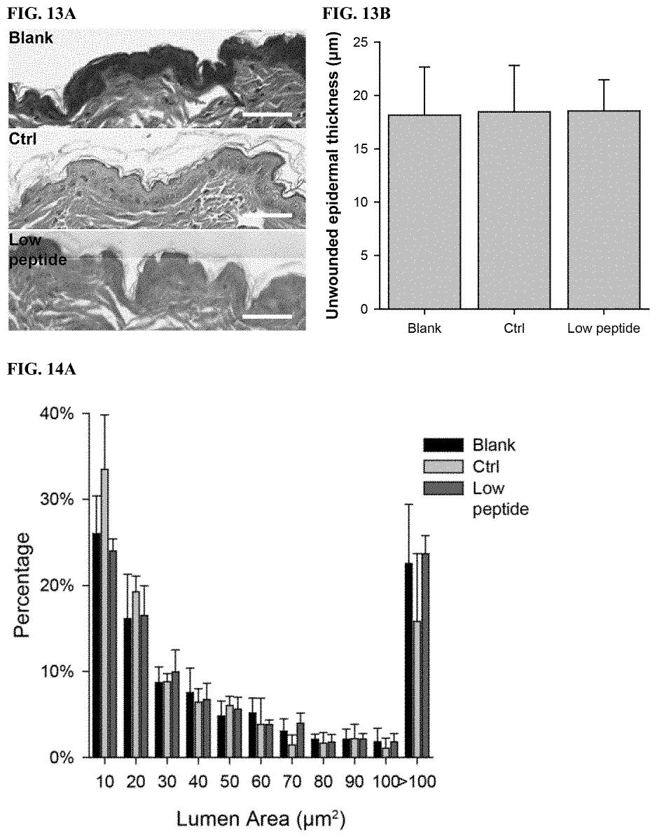

The wound histology was examined by Masson's trichrome staining and confirmed the location of the epithelial tongue using pan-keratin staining (FIG. 4C). The wound edge was defined as the distance between the two boundaries of intact skin (thin musculature of the Panniculus carnosus). There was no significant difference amongst the three groups in the wound edge distance, indicating no difference in wound contraction over 14 days (FIG. 4Di). The epithelial gap was defined as the distance between the two advancing epithelial tongues (FIG. 4Ci-vi) and the epithelial gap in the Low Peptide group was smaller than in the Blank and Ctrl groups (FIG. 4Dii, FIG. 12). The re-epithelialization percentage was defined as the ratio of the distance that has been re-epithelialized over the wound edge distance, and the re-epithelialization percentage was significantly higher in the presence of the QHREDGS peptide compared to the controls (FIG. 4Diii, FIG. 12). The Low Peptide group also developed significantly more granulation tissue (FIG. 4Div) compared to the controls. Moreover, the epidermal thickness of the advancing epithelial tongue was significantly smaller in the Peptide group than in the Blank and Ctrl groups (FIG. 4Dv). There was no difference in the epithelial thickness of the skin remote from the wounds among the three experimental groups (FIG. 13).

QHREDGS-Hydrogel Increases Total Blood Vessel Count in the Wound

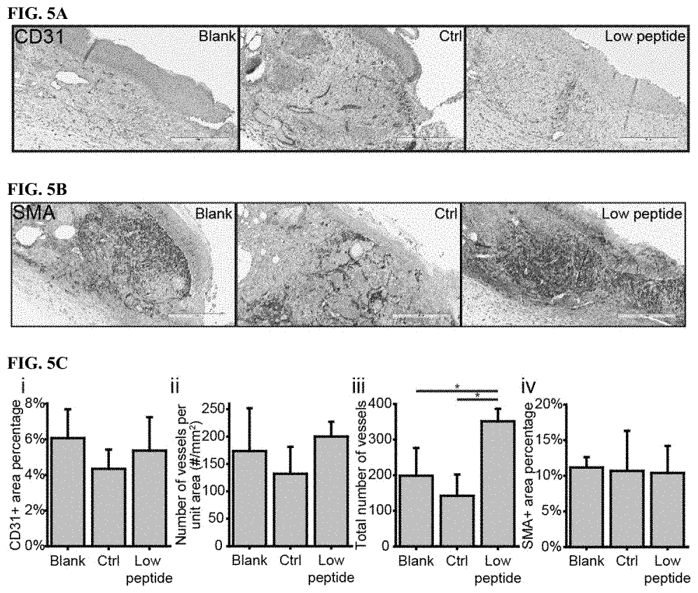

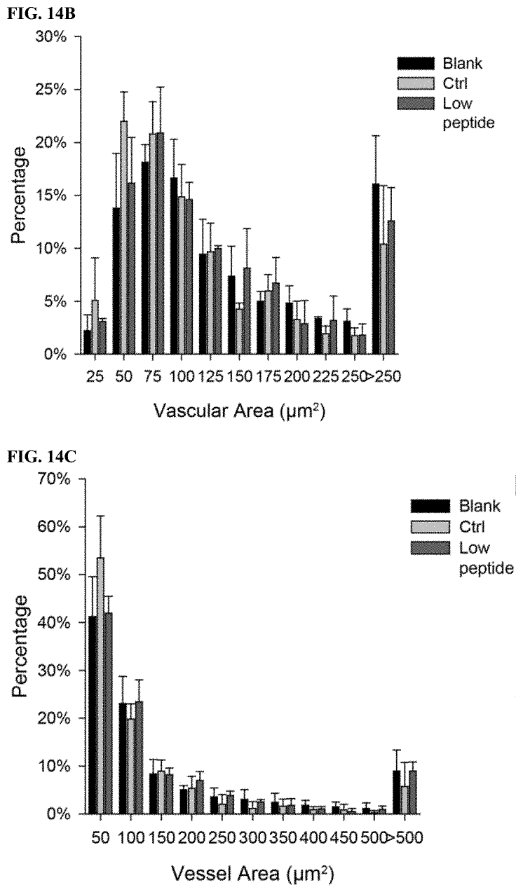

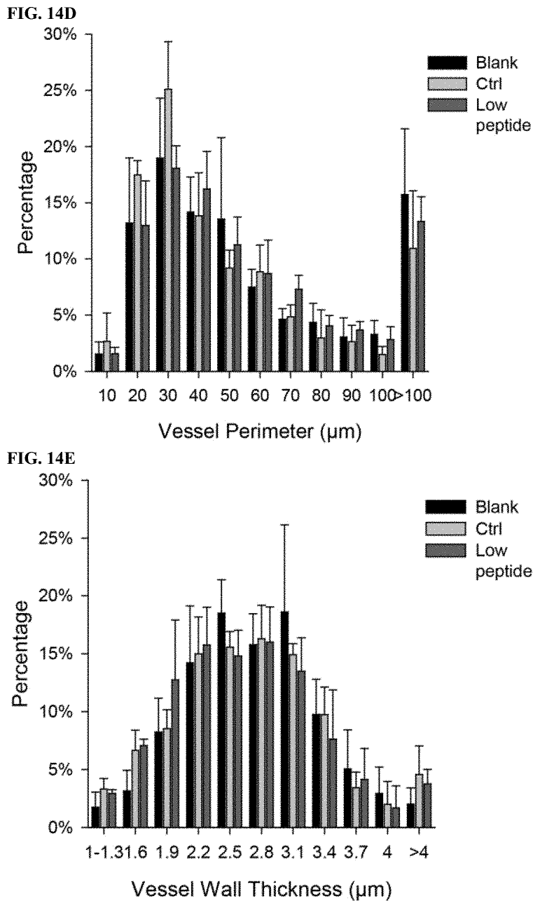

To further characterize the granulation tissue, the density of microvessels and contracting myofibroblasts was compared in the three experimental groups by immunohistochemistry with antibodies against CD31 (FIG. 5A) and α-smooth muscle actin (α-SMA) (FIG. 5B). There was no difference in the vessel density and CD31 positive area percentage among the three groups based on measurements obtained from six random locations within the granulation tissue (FIG. 5C). This was further confirmed by an automated algorithm analysis of the entire granulation tissue that showed no significant difference amongst the three groups in terms of vessel density, CD31 positive percentage, lumen area, vascular area, vessel area, vessel perimeter, or vessel wall thickness (FIG. 14). However, there was an effect on the total blood vessel count in the wound. The total number of blood vessels significantly increased in the Low Peptide group compared to the controls (FIG. 5Ciii), which is related to the increased granulation tissue formation in the Low Peptide group compared to the controls (FIG. 4Div). There was also no significant difference in the density of myofibroblasts amongst the three groups as determined by α-SMA staining (a common, albeit non-specific, marker of myofibroblasts) (FIG. 5Civ). Myofibroblasts are considered to be the main contributors to wound contraction. Taken together, the accelerated wound healing in the presence of the conjugated QHREDGS peptide cannot be attributed to changes in granulation tissue blood vessel density or to myofibroblasts, but possibly to more granulation tissue overall in the Low Peptide group (FIG. 4Div).

High Peptide Hydrogel Outperforms a Clinically-Approved Wound Dressing in Wound Closure of Db/Db Diabetic Mice

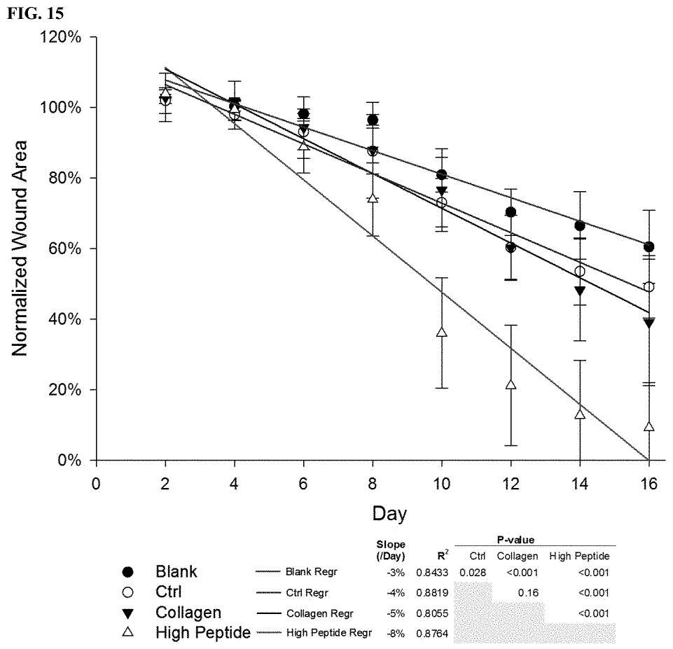

To demonstrate the full potential of the peptide-modified hydrogels and to benchmark against an FDA approved and clinically-available treatment, a High Peptide hydrogel (High Peptide) was compared to ColActive® collagen dressing (Collagen) in vivo. After 21 days, a visual inspection indicated essentially closed wounds in animals in the High Peptide group (FIG. 6A). Upon sacrificing the animals, analysis of wound images indicated 100% wound closure in 3/5 animals in the High Peptide group, 1/5 in the ColActive® (Collagen) group and 0/5 in the Blank and the peptide-free hydrogel (Ctrl) controls. The advantages of the high dose Peptide hydrogel treatment became apparent at day 8 after treatment (FIG. 6B), and persisted throughout the healing period up to day 21, at which point the wounds in the High Peptide group were essentially closed. In the linear range of wound closure (FIG. 6B day2-16), the fitted line slopes indicated a significantly higher wound closure rate in the High Peptide group (8% of wound area/day) compared to the Blank (3%/day), peptide-free hydrogel (Ctrl; 4%/day) and ColActive® dressing (Collagen; 5%/day) groups (FIG. 15). There was no significant difference in the wound closure rate between the peptide-free hydrogel (Ctrl) and the ColActive® dressing, however the rate for the High Peptide hydrogel was significantly faster than all other groups (FIG. 15).

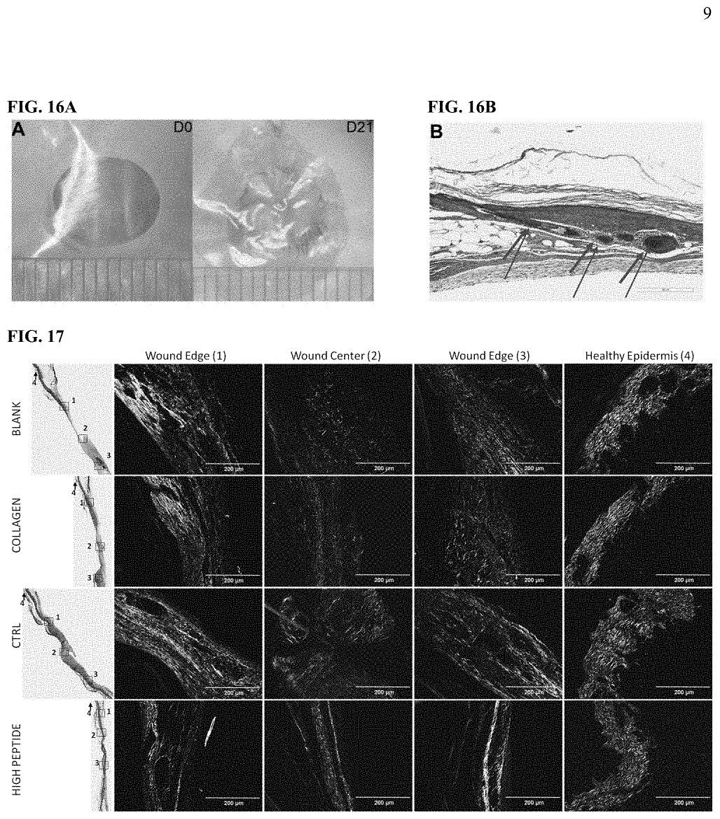

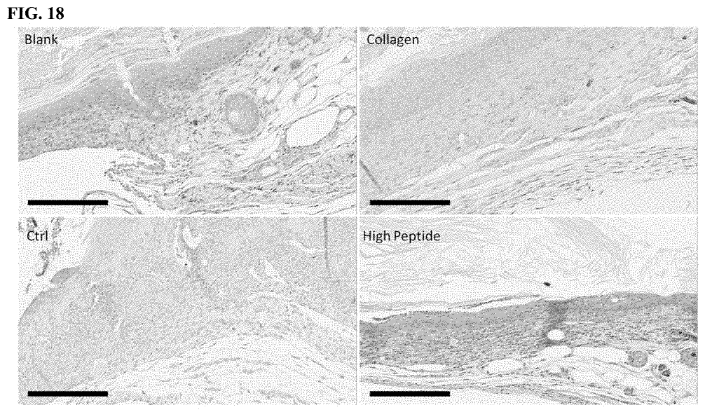

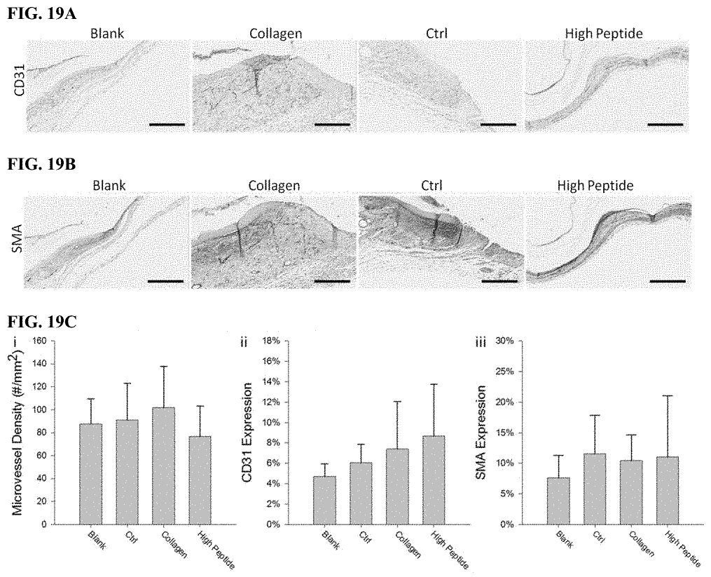

Histological analysis (FIG. 6C) indicated that only the High Peptide group animals had a significant difference in the wound edge distance (FIG. 6Di), epithelial gap (FIG. 6Dii), re-epithelialization percentage (FIG. 6Diii) and the epithelial thickness (FIG. 6Div) relative to the Blank. In High Peptide group wounds, the epithelial thickness was also comparable to unwounded epidermis (FIG. 6Div, FIG. 13B) whereas the other treatment groups had significantly (>2 times) thicker epithelium than the unwounded epidermis. According to these histological measures of wound closure and epidermis quality, the High Peptide group significantly outperformed both the peptide-free hydrogel (Ctrl) and the ColActive® dressing (Collagen) (FIG. 6Dii-iv). Notably, in the High Peptide group, one mouse with complete wound closure also exhibited hair regrowth at the wound periphery as seen in gross morphology (FIG. 16A) and histology (FIG. 16B) images. Picrosirius red staining indicated disruption of the native collagen organization in day 21 wounds in Blank, peptide-free hydrogel (Ctrl) and ColActive® dressing (Collagen) treated mice (FIG. 17). In contrast, collagen organization equivalent to the unwounded skin was identified in wounds treated with the High Peptide-immobilized hydrogel (FIG. 17). Staining for neural marker PgP9.5 confirmed a low density of positive cells in all groups, with no appreciable differences between groups (FIG. 18). This is consistent with the inability of diabetic mice to fully regenerate neurons. There were no significant differences in the vascular density amongst the groups at day21 (FIG. 19).

To further delineate if there were differences in blood vessel densities in the early stages of healing, a time-course of early vessel development in the High Peptide and Blank groups was compared. There were no appreciable differences in blood vessel density at Day 4 and 8 after injury, in the High Peptide compared to the Blank group (FIG. 19). It was, however, observed that an increase in vessel density and CD31+ area between Day 4 and 8 in the High Peptide group but not in the Blank group (FIG. 19). Blood vessel density and percent area covered by endothelial cells remained statistically unchanged between Day 8 and Day 21 (FIG. 19) according to ANOVA analysis. Collectively these results indicate that the treatment with High-peptide hydrogel lead to sufficient angiogenesis to drive rapid granulation tissue formation, without massive vessel overgrowth early on, followed by a pronounced vessel regression, as it is often reported with treatments that overstimulate early angiogenesis.

Pre-Gelled Hydrogel Patch

In certain aspects, the present disclosure provides a pre-gelled hydrogel patch comprising the hydrogel-peptide composition as described herein, wherein the hydrogel patch is applied to the skin or wound.

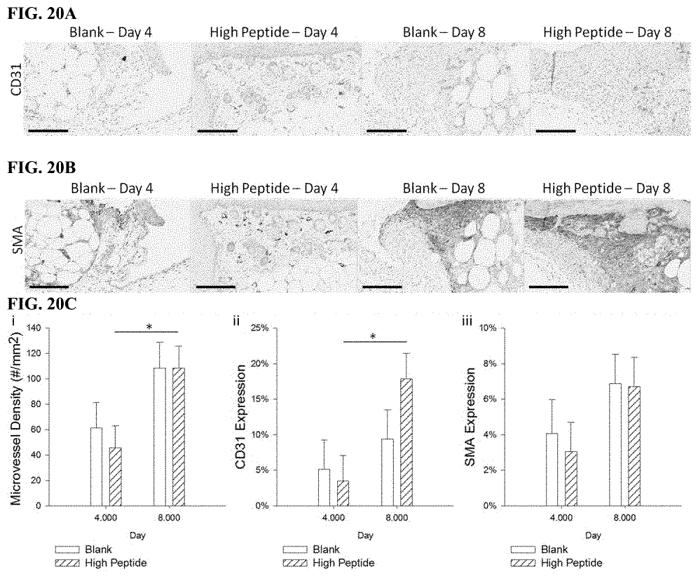

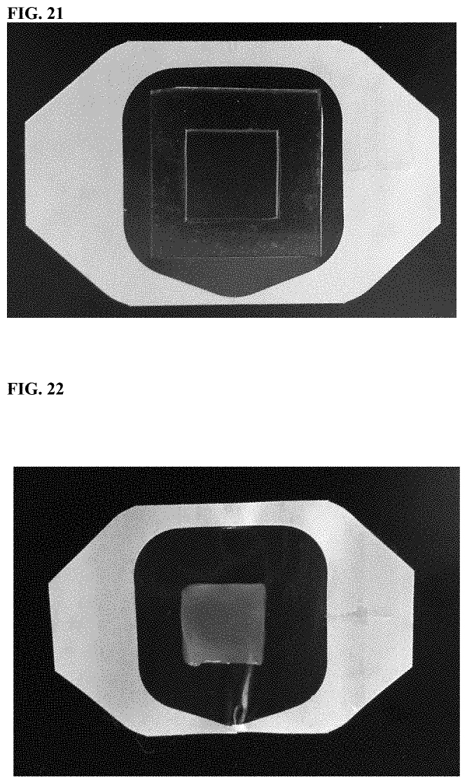

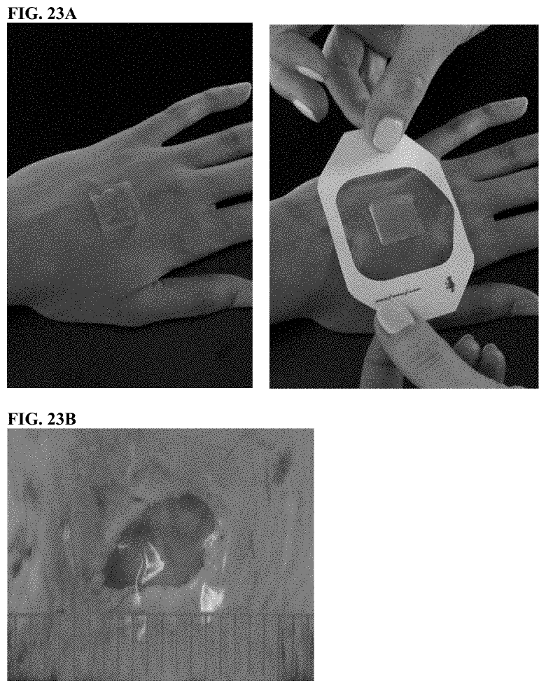

Q-peptide modified hydrogel for pre-gelled patch application is prepared with the same materials and using the same chemistry as the hydrogel for the fresh, liquid solution application. As a first step, QHREDGS peptide is conjugated to chitosan using EDC/S-NHS chemistry, and the conjugated chitosan is lyophilized. The lyophilized chitosan is dissolved overnight at 4° C. in 0.9% normal saline and type 1 collagen, at a 1:1 ratio, to obtain a concentration of 3.62 mg/mL. The chitosan-collagen solution is neutralized using 0.1N NaOH, and diluted to a final concentration of 2.5 mg/mL. For the pre-gelled application, Tegaderm™, a transparent medical dressing adhesive, is unwrapped, and a mold created using PDMS is placed on top (FIG. 20a). The neutralized peptide hydrogel prepared by the protocol above is cast in the PDMS mold on the Tegaderm™, and crosslinked at 37° C. for 1 hour. The PDMS mold is removed, and the Tegaderm plus pre-gelled peptide hydrogel patch is obtained (FIG. 20b). The hydrogel patch can be placed on skin for minor cuts, scrapes and abrasions (FIG. 20c) or on wounds such as surgical wounds, burns, chronic wounds (FIG. 20d). The remaining wrapper on the Tegaderm™ can be peeled away.

The hydrogel patch on the Tegaderm™ can also be sealed and stored at 37° C., with a relative humidity of 95% and 5% CO2 for later use.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

1. A method of treating an epithelial wound in a patient in need there of, the method comprising administering topically to the epithelial wound of the patient a formulation comprising a biomaterial and at least one peptide of the formula:
X₁ X₂ X₃ X₄X₅X₆ X₇ wherein:
X₁ is an optional residue selected from glutamine, threonine, serine or asparagine;
X₂ is an optional positively charged residue selected from histidine, arginine or lysine;
X₃ is glutamate, threonine, isoleucine, histidine, lysine, glutamine, tyrosine, valine or leucine;
X₄ is glycine or valine;
X₅ is an optional residue selected from serine, threonine, aspartic acid, isoleucine or glycine;
X₆ is an optional residue selected from leucine, valine, glutamine,
glycine, isoleucine or serine; and
X₇ is an optional residue selected from aspartic acid, asparagine, valine or lysine; and wherein the method is effective for treating the epithelial wound.

2. The method of claim 1, wherein the peptide is immobilized to the biomaterial.

3. The method of claim 2, wherein the biomaterial is a hydrogel.

4. The method of claim 1, wherein the epithelial wound is a sore, a cold sore, a cutaneous opening, a lesion, an abrasions, a burn, a rash, an ulcer, a pressure ulcer, an arterial ulcer, a venous ulcer, a diabetes-related wound, a burn, a sun burn, an aging skin wound, a corneal ulceration wound, an inflammatory gastrointestinal tract disease wound, a bowel inflammatory disease wound, a Crohn's disease wound, an ulcerative colitis, a hemorrhoid, an epidermolysis bulosa wound, a skin blistering wound, a psoriasis wound, an animal skin wound, a proud flesh wound, an animal diabetic wound, a retinopathy wound, an oral wound (mucositis), a vaginal mucositis wound, a gum disease wound, a laceration, a surgical incision wound, a post-surgical adhesions wound, a grafted skin site or a donor skin site.

5. The method of claim 1, wherein the epithelial wound is an external wound.

6. The method of claim 3, wherein the hydrogel comprises at least one biomaterial and a solvent.

7. The method of claim 6, wherein the solvent is water.

8. The method of claim 3, wherein the hydrogel is a polyacrylic acid hydrogel, a povidone hydrogel or a cellulose hydrogel.

9. The method of claim 3, wherein the hydrogel comprises at least one of chitosan, alginate, agarose, methylcellulose, hyaluronan, collagen, laminin, matrigel, fibronectin, vitronectin, poly-l-lysine, proteoglycans, fibrin glue, gels made by decellularization of engineered and natural tissues, and a combination thereof.

10. The method of claim 3, wherein the hydrogel comprises at least one of polyglycolic acid (PGA), polylactic acid (PLA) and combinations of PGA and PLA such as PLGA, poly F-caprolactone, polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), self-assembling peptide hydrogels, AcN-RARADADARARADADA-CNH (SEQ ID NO.2), polyurethanes, poly(isopropylacrylamide), poly(N-isopropylacrylamide), [poly(NIPAM)] or a combination thereof.

11. The method of claim 3, wherein the hydrogel has an average molecular weight of about 100 Daltons (Da) to about 1,000,0001 Da.

12. The method of claim 3, wherein the hydrogel has a viscosity from about 100 to about 10,000 cps.

13. The method of claim 1, wherein the formulation further comprises at least one stabilizer.

14. The method of claim 1, wherein the at least one peptide is at least one of: QHREDGS (SEQ ID NO.:1), REDG (SEQ ID NO.: 3), RLDG (SEQ ID NO.: 4), REDGS (SEQ ID NO.: 5), RLDGS (SEQ ID NO.: 6), HREDG (SEQ ID NO.: 7), HRLDG (SEQ ID NO.: 8), HREDGS (SEQ ID NO.: 9), HRLDGS (SEQ ID NO.; 10), QHREDG (SEQ ID NO.: 11), QHRLDG (SEQ ID NO.: 12), QHREDVS (SEQ ID NO.: 13), KRLDGS (SEQ ID NO.: 16), QHREDGSL (SEQ ID NO.: 17), QHRLDGSL (SEQ ID NO 18), QHRLDGSLD (SEQ ID NO.: 19), QHREDGSLD (SEQ ID NO.: 20), or a combination thereof.

15. The method of claim 14, wherein the at least one peptide is QHREDGS (SEQ ID NO.:1).

16. The method of 15, wherein the QHREDGS (SEQ ID NO.:1) peptide is present in the biomaterial is at a concentration of about 75 μM to about 750 μM.

17. The method of claim 16, wherein the QHREDGS (SEQ ID NO.:1) peptide is present in the biomaterial at a concentration of about 100 μM to about 600 μM.

18. The method of claim 16, wherein the QHREDGS (SEQ ID NO.:1) peptide is present in the biomaterial in a concentration of about 150 μM to about 500 μM.

19. The method of claim 3, wherein the QHREDGS (SEQ ID NO.:1) peptide is conjugated to the hydrogel.

20. A method of treating an epithelial wound in a patient in need thereof, the method comprising administering topically to the epithelial wound of said patient a formulation comprising a hydrogel and a peptide having the sequence QHREDGS (SEQ ID NO.:1), wherein the peptide is immobilized to the hydrogel, and wherein the method is effective for treating the epithelial wound.


SEQUENCE LISTING (20 sequences)

SEQ ID NO: 1 - protein, 7 aa, Artificial Sequence
  Synthetic peptide
QHREDGS

SEQ ID NO: 2 - protein, 16 aa, Artificial Sequence
  Synthetic sequence
RARADADARARADADA

SEQ ID NO: 3 - protein, 4 aa, Artificial Sequence
  Synthetic sequence
REDG

SEQ ID NO: 4 - protein, 4 aa, Artificial Sequence
  Synthetic sequence
RLDG

SEQ ID NO: 5 - protein, 5 aa, Artificial Sequence
  Synthetic sequence
REDGS

SEQ ID NO: 6 - protein, 5 aa, Artificial Sequence
  Synthetic sequence
RLDGS

SEQ ID NO: 7 - protein, 5 aa, Artificial Sequence
  Synthetic sequence
HREDG

SEQ ID NO: 8 - protein, 5 aa, Artificial Sequence
  Synthetic sequence
HRLDG

SEQ ID NO: 9 - protein, 6 aa, Artificial Sequence
  Synthetic sequence
HREDGS

SEQ ID NO: 10 - protein, 6 aa, Artificial Sequence
  Synthetic sequence
HRLDGS

SEQ ID NO: 11 - protein, 6 aa, Artificial Sequence
  Synthetic sequence
QHREDG

SEQ ID NO: 12 - protein, 6 aa, Artificial Sequence
  Synthetic sequence
QHRLDG

SEQ ID NO: 13 - protein, 7 aa, Artificial Sequence
  Synthetic sequence
QHREDVS

SEQ ID NO: 14 - protein, 7 aa, Artificial Sequence
  Synthetic sequence
QHREDGS

SEQ ID NO: 15 - protein, 7 aa, Artificial Sequence
  Synthetic sequence
QHRLDGS

SEQ ID NO: 16 - protein, 6 aa, Artificial Sequence
  Synthetic sequence
KRLDGS

SEQ ID NO: 17 - protein, 8 aa, Artificial Sequence
  Synthetic sequence
QHREDGSL

SEQ ID NO: 18 - protein, 8 aa, Artificial Sequence
  Synthetic sequence
QHRLDGSL

SEQ ID NO: 19 - protein, 9 aa, Artificial Sequence
  Synthetic sequence
QHRLDGSLD

SEQ ID NO: 20 - protein, 9 aa, Artificial Sequence
  Synthetic sequence
QHREDGSLD